(19) United States
(12) Patent Application Publication
(10) Pub. No.: US 2004/0002114 A1
(43) Pub. Date: Jan. 1, 2004

(54) Nucleic acids encoding a G-protein coupled receptor involved in islet cell signaling

(75) Inventors: Francine M. Gregoire, Lafayette, CA (US); Jeffrey D. Johnson, Moraga, CA (US); John E. Blume, Danville, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: METABOLEX, INC., Hayward, CA (US)

(21) Appl. No.: 10/328,916

(22) Filed: Jan. 6, 2003

Related U.S. Application Data
(60) Provisional application No. 60/345,697, filed on Jan. 4, 2002.

(51) Int. Cl. G01N 33/53; A61K 38/17; C07K 14/72; C12P 21/02; C12N 5/06; C07H 21/04

(52) U.S. Cl. 435/7.1; 435/69.1; 435/320.1; 435/325; 530/350; 536/23.5; 514/12

(57) ABSTRACT

The invention provides isolated nucleic acid and amino acid sequences of human and murine islet cell G-protein coupled receptors, antibodies to such receptors, methods of detecting such nucleic acids and receptors, methods of screening for modulators of islet cell G-protein coupled receptors and methods of treating mammals with modulators of islet cell G-protein coupled receptor activity.

### PRIORITY

This application claims the benefit of priority under 35 U.S.C. §119(e) to U.S. Provisional Application No. 60/345,697 filed Jan. 4, 2002, which is incorporated herein by reference in its entirety and for all purposes.

### FIELD OF THE INVENTION

The invention provides isolated nucleic acid and amino acid sequences of G-protein coupled receptors involved in islet cell signaling, antibodies to such receptors, methods of detecting such nucleic acids and receptors, methods of screening for modulators of G-protein coupled receptor signaling in islet cells, and methods of treating mammals with modulators of islet cell G-protein coupled receptor activity.

### BACKGROUND OF THE INVENTION

G-protein coupled receptors (GPCRs) are the largest family of cell surface molecules involved in signal transmission. These receptors form a class of polypeptides containing seven transmembrane domains which transduce an extracellular signal into a cellular response. These receptors are activated by a wide variety of ligands, including peptide and non-peptide neurotransmitters, hormones, growth factors, odorant molecules and light. GPCRs are encoded by the largest gene family in most animal genomes. For example, more than 1% of the human genome encodes greater than 1000 proteins with the characteristic heptahelical structure (Flower, Biochem. Biophys. Acta 1422: 207-234, (1999)). The physiological importance of GPCRs is supported by numerous GPCR knockout animal studies (Rohrer et al., Physiol. Rev., 78: 35-52 (1998)) and by their linkage to hereditary diseases (Stadel et al., Trends Pharmacol. Sci., 18:430-437 (1997)).

The animal pancreas is composed of an exocrine portion and an endocrine portion. The exocrine portion comprises 80-85% of the organ and is made up of numerous small glands (acini) containing columnar to pyramidal epithelial cells radially arrayed about the gland circumference. The exocrine portion of the pancreas secretes on average 2-2.5 liters per day of a bicarbonate-rich fluid containing digestive enzymes and proenzymes. Regulation of the exocrine secretion involves neural stimulation by the vagus nerve and hormonal stimulation.

The endocrine portion of the animal pancreas consists of approximately 1 million microscopic clusters of cells, the islet cells, which comprise 0.7-2.5% of the weight of the organ. The islet cells consist of 4 major and 2 minor cell types. The four major cell types are the β, α, δ and PP (pancreatic polypeptide) cells which comprise 68%, 20%, 10%, and 2% respectively of the adult islet cell population. The two rare cell types consist of D1 cells and enterochromaffin cells. Each cell type can be differentiated morphologically by their staining properties, by the ultrastructural structure of their intracellular granules and by their hormone content. β cells secrete insulin, which modulates carbohydrate and lipid metabolism and influences the biosynthesis of protein and RNA. α cells secrete glucagon, which modulates glycogen metabolism. δ cells secrete somatostatin which suppresses both insulin and glucagon release and suppresses release of somatotropin from the hypothalamus. PP cells secrete a unique pancreatic polypeptide which stimulates the secretion of gastric and intestinal enzymes and inhibits intestinal motility. D1 cells secrete vasoactive intestinal peptide (VIP), a hormone that regulates glycogen metabolism and hyperglycemia. Enterochromaffin cells secrete serotonin. Each cell type releases their respective hormones in response to multiple extracellular stimuli. For example, β cells are induced to secrete insulin in response to glucose uptake and in response to stimulation by intestinal hormones, certain amino acids and sulfonylureas. A recent study indicated that insulin secretion can be induced via a GPCR signal transduction mechanism in a calcium independent manner (Lang et al., EMBO J., 17:648-657 (1998)). The major physiological disorder of islet cells is diabetes mellitus.

Diabetes mellitus can be divided into two clinical syndromes, Type I and Type II diabetes mellitus. Type I, or insulin-dependent diabetes mellitus (IDDM), is a chronic autoimmune disease characterized by the extensive loss of beta cells. As these cells are progressively destroyed, the amount of secreted insulin decreases, eventually leading to hyperglycemia (abnormally high level of glucose in the blood) when the amount secreted insulin drops below the level required for euglycemia (normal blood glucose level). Although the exact trigger for this immune response is not known, patients with IDDM have high levels of antibodies against pancreatic beta cells. However, not all patients with high levels of these antibodies develop IDDM.

Type II diabetes (also referred to as non-insulin dependent diabetes mellitus (NIDDM)) develops when muscle, fat and liver cells fail to respond normally to insulin. This failure to respond (called insulin resistance) may be due to reduced numbers of insulin receptors on these cells, or a dysfunction of signaling pathways within the cells, or both. The beta cells initially compensate for this insulin resistance by increasing their insulin output. Over time, these cells become unable to produce enough insulin to maintain normal glucose levels, indicating progression to type II diabetes.

Type II diabetes is brought on by a combination of poorly understood genetic and acquired risk factors—including a high-fat diet, lack of exercise, and aging. Worldwide, type II diabetes has become an epidemic, driven by increases in obesity and a sedentary lifestyle, widespread adoption of western dietary habits, and the general aging of the populations in many countries. In 1985, an estimated 30 million people worldwide had diabetes—by 2000, this figure had increased 5-fold, to an estimated 154 million people. The number of people with diabetes is expected double between now and 2025, to about 300 million.

Type II diabetes is a complex disease characterized by defects in glucose and lipid metabolism. Typically there are perturbations in many metabolic parameters including increases in fasting plasma glucose levels, free fatty acid levels and triglyceride levels, as well as a decrease in the ratio of HDL/LDL. As discussed above, one of the principal underlying causes of diabetes is thought to be an increase in insulin resistance in peripheral tissues, principally muscle and fat.

There is no cure for diabetes. Conventional treatments for diabetes are very limited, and focus on attempting to control blood glucose levels in order to minimize or delay complications.

### SUMMARY OF THE INVENTION

The present invention thus provides for the first time nucleic acids encoding human and murine islet cell G-protein coupled receptors. These nucleic acids and the polypeptides that they encode are referred to as IC-GPCR for islet cell G-protein coupled receptor. These islet cell GPCRs are components of the islet cell signal transduction pathway.

In one aspect, the present invention provides an isolated nucleic acid encoding an islet cell signal transduction G-protein coupled receptor, the receptor comprising greater than 70% amino acid identity to an amino acid sequence of SEQ ID NO:1, or SEQ ID NO:2.

In another aspect, the present invention provides an isolated nucleic acid encoding an islet cell signal transduction G-protein coupled receptor, wherein the nucleic acid specifically hybridizes under highly stringent conditions to a nucleic acid having the sequence of SEQ ID NO:3, or SEQ ID NO:4.

In another aspect, the present invention provides an isolated nucleic acid encoding an islet cell signal transduction G-protein coupled receptor, the receptor comprising greater than 70% amino acid identity to a polypeptide having a sequence of SEQ ID NO:1, or SEQ ID NO:2, wherein the nucleic acid selectively hybridizes under moderately stringent hybridization conditions to a nucleotide sequence of SEQ ID NO:3, or SEQ ID NO:4.

In another aspect, the present invention provides an isolated nucleic acid encoding an extracellular domain of an islet cell signal transduction G-protein coupled receptor, the extracellular domain having greater than 70% amino acid sequence identity to the extracellular domain of SEQ ID NO:1 or SEQ ID NO:2.

In another aspect, the present invention provides an isolated nucleic acid encoding a transmembrane domain of an islet cell signal transduction G-protein coupled receptor, the transmembrane domain comprising greater than 70% amino acid sequence identity to the transmembrane domain of SEQ ID NO:1 or SEQ ID NO:2.

In another aspect, the present invention provides an isolated islet cell signal transduction G-protein coupled receptor, the receptor comprising greater than about 70% amino acid sequence identity to an amino acid sequence of SEQ ID NO:1, or SEQ ID NO:2.

In one embodiment, the receptor specifically binds to polyclonal antibodies generated against SEQ ID NO:1, or SEQ ID NO:2. In another embodiment, the receptor has G-protein coupled receptor activity. In another embodiment, the receptor has an amino acid sequence of SEQ ID NO:1, or SEQ ID NO:2. In another embodiment, the receptor is from a human, or a murine.

In one aspect, the present invention provides an isolated polypeptide comprising an extracellular domain of a islet cell signal transduction G-protein coupled receptor, the extracellular domain comprising greater than about 70% amino acid sequence identity to the extracellular domain of SEQ ID NO:1 or SEQ ID NO:2.

In one embodiment, the polypeptide encodes the extracellular domain of SEQ ID NO:1 or SEQ ID NO:2. In another embodiment, the extracellular domain is covalently linked to a heterologous polypeptide, forming a chimeric polypeptide.

In one aspect, the present invention provides an isolated polypeptide comprising a transmembrane domain of an islet cell signal transduction G-protein coupled receptor, the transmembrane domain comprising greater than about 70% amino acid sequence identity to the transmembrane domain of SEQ ID NO:1 or SEQ ID NO:2.

In one embodiment, the polypeptide encodes the transmembrane domain of SEQ ID NO:1 or SEQ ID NO:2. In another embodiment, the polypeptide further comprises a cytoplasmic domain comprising greater than about 70% amino acid identity to the cytoplasmic domain of SEQ ID NO:1 or SEQ ID NO:2. In another embodiment, the polypeptide encodes the cytoplasmic domain of SEQ ID NO:1 or SEQ ID NO:2. In another embodiment, the transmembrane domain is covalently linked to a heterologous polypeptide, forming a chimeric polypeptide. In another embodiment, the chimeric polypeptide has G-protein coupled receptor activity.

In one aspect, the present invention provides an antibody that selectively binds to the receptor comprising greater than about 70% amino acid sequence identity to an amino acid sequence of SEQ ID NO:1, or SEQ ID NO:2.

In another aspect, the present invention provides an expression vector comprising a nucleic acid encoding a polypeptide comprising greater than about 70% amino acid sequence identity to an amino acid sequence of SEQ ID NO:1 or SEQ ID NO:2.

In another aspect, the present invention provides a host cell transfected with the expression vector.

In another aspect, the present invention provides a method for identifying a compound that modulates receptor signaling in islet, cells, the method comprising the steps of: (i) contacting the compound with a polypeptide comprising an extracellular domain of a signal transduction G-protein coupled receptor, the extracellular domain comprising greater than about 70% amino acid sequence identity to the extracellular domain of SEQ ID NO:1, or SEQ ID NO:2; and (ii) determining the functional effect of the compound upon the extracellular domain.

In another aspect, the present invention provides a method for identifying a compound that modulates receptor signaling in islet cells, the method comprising the steps of: (i) contacting the compound with a polypeptide comprising an extracellular domain of an islet cell signal transduction G-protein coupled receptor, the transmembrane domain comprising greater than about 70% amino acid sequence identity to the transmembrane domain of SEQ ID NO:1, or SEQ ID NO:2; and (ii) determining the functional effect of the compound upon the transmembrane domain.

In one embodiment, the polypeptide is an islet cell signal transduction G-protein coupled receptor, the receptor comprising greater than about 70% amino acid identity to a polypeptide encoding SEQ ID NO:1, or SEQ ID NO:2. In another embodiment, polypeptide comprises an extracellular domain that is covalently linked to a heterologous polypeptide, forming a chimeric polypeptide. In another embodiment, the polypeptide has G-protein coupled receptor activity. In another embodiment, the extracellular domain is linked to a solid phase, either covalently or non-covalently. In another embodiment, the functional effect is determined by measuring changes in intracellular cAMP, IP3, or Ca²⁺. In another embodiment, the functional effect is a chemical effect. In another embodiment, the functional effect is insulin release; glucagon release; somatostatin release; pancreatic polypeptide release; VIP release or serotonin release. In another embodiment, the functional effect is determined by measuring binding of the compound to the extracellular domain. In another embodiment, the polypeptide is recombinant. In another embodiment, the polypeptide is expressed in a cell or cell membrane. In another embodiment, the cell is a eukaryotic cell.

In one embodiment, the polypeptide comprises an transmembrane domain that is covalently linked to a heterologous polypeptide, forming a chimeric polypeptide.

In one aspect, the present invention provides a method of making an islet cell signal transduction G-protein coupled receptor, the method comprising the step of expressing the receptor from a recombinant expression vector comprising a nucleic acid encoding the receptor, wherein the amino acid sequence of the receptor comprises greater than 70% amino acid identity to a polypeptide having a sequence of SEQ ID NO:1, or SEQ ID NO:2.

In one aspect, the present invention provides a method of making a recombinant cell comprising an islet cell signal transduction G-protein coupled receptor, the method comprising the step of transducing the cell with an expression vector comprising a nucleic acid encoding the receptor, wherein the amino acid sequence of the receptor comprises greater than about 70% amino acid identity to a polypeptide having a sequence of SEQ ID NO:1, or SEQ ID NO:2.

In one aspect, the present invention provides a method of making a recombinant expression vector comprising a nucleic acid encoding an islet cell signal transduction G-protein coupled receptor, the method comprising the step of ligating to an expression vector a nucleic acid encoding the receptor, wherein the amino acid sequence of the receptor comprises greater than about 70% amino acid identity to a polypeptide having a sequence of SEQ ID NO:1, or SEQ ID NO:2.

### DEFINITIONS

As used herein, the following terms have the meanings ascribed to them unless specified otherwise.

“IC-GPCR,” refers to a G-protein coupled receptor that is specifically expressed in islet cells of the pancreas. IC-GPCR has the ability to act as a receptor for signal transduction in islet cells.

IC-GPCR encodes GPCRs with seven transmembrane regions that have “G-protein coupled receptor activity,” e.g., they bind to G-proteins in response to extracellular stimuli and promote production of second messengers such as IP3, cAMP, and Ca²⁺ via stimulation of enzymes such as phospholipase C and adenylate cyclase (for a description of the structure and function of GPCRs, see, e.g., Fong, supra, and Baldwin, supra).

The term IC-GPCR therefore refers to polymorphic variants, alleles, mutants, and interspecies homologs that: (1) have about 70% amino acid sequence identity, optionally about 75, 80, 85, 90, or 95% amino acid sequence identity to SEQ ID NOs:1 and 2 over a window of about 25 amino acids, optionally 50-100 amino acids; (2) bind to antibodies raised against an immunogen comprising an amino acid sequence selected from the group consisting of SEQ ID NOs:1 and 2 and conservatively modified variants thereof; (3) specifically hybridize (with a size of at least about 500, optionally at least about 900 nucleotides) under stringent hybridization conditions to a sequence selected from the group consisting of SEQ ID NOs:3 and 4, and conservatively modified variants thereof; or (4) are amplified by primers that specifically hybridize under stringent hybridization conditions to sequences identified in SEQ ID NOs:3 and 4.

Topologically, sensory GPCRs have an N-terminal extracellular domain, a “transmembrane domain” comprising seven transmembrane regions and corresponding cytoplasmic and extracellular loops, and a C-terminal “cytoplasmic domain” (see, e.g., Hoon et al., Cell 96:541-551 (1999); Buck & Axel, Cell 65:175-187 (1991)). These domains can be structurally identified using methods known to those of skill in the art, such as sequence analysis programs that identify hydrophobic and hydrophilic domains (see, e.g., Kyte & Doolittle, J. Mol. Biol. 157:105-132 (1982)). Such domains are useful for making chimeric proteins and for in vitro assays of the invention.

“Extracellular domain” therefore refers to the N-terminal extracellular domain of the human and murine IC-GPCR that protrudes from the cellular membrane. The N-terminal extracellular domain of the human IC-GPCR starts at the N-terminus and ends approximately at amino acid 71 of SEQ ID NO:1. The region corresponding to amino acids 1-71 of SEQ ID NO:1. The N-terminal extracellular domain of the murine IC-GPCR starts at the N-terminus and ends approximately at amino acid 49. The region corresponding to amino acids 1-49 of SEQ ID NO:2. These embodiment are useful for in vitro ligand binding assays, both soluble and solid phase.

“Transmembrane domain,” comprising seven transmembrane regions plus the corresponding cytoplasmic and extracellular loops, refers to the domain of the human IC-GPCR that starts approximately at amino acid 72 and ends approximately at amino acid 328 of SEQ ID NO:1. The transmembrane domain of the murine IC-GPCR starts approximately at amino acid 50 and ends approximately at amino acid 307 of SEQ ID NO:2.

The “cytoplasmic domain” refers to the domain of the human IC-GPCR that starts at approximately amino acid 329 of SEQ ID NO:1 and continues to the C-terminus of the polypeptide. The cytoplasmic domain of the murine IC-GPCR begins at approximately amino acid 308 of SEQ ID NO:2 and continues to the C-terminus of the peptide.

The “ligand binding domain” refers to that portion of the human and murine IC-GPCR which binds ligand. The ligand binding domain may be a portion of the N-terminal extracellular domain or, alternatively, may be a portion of the extracellular loops separating the membrane spanning portions of the transmembrane domain.

“Biological sample” as used herein is a sample of biological tissue or fluid that contains IC-GPCR or nucleic acid encoding IC-GPCR protein. Such samples include, but are not limited to, tissue isolated from humans, mice, and rats, in particular, pancreas. Biological samples may also include sections of tissues such as frozen sections taken for histological purposes. A biological sample is typically obtained from a eukaryotic organism, such as insects, protozoa, birds, fish, reptiles, and preferably a mammal such as rat, murine, cow, dog, guinea pig, or rabbit, and most preferably a primate such as chimpanzees or humans. Tissues include pancreas, spleen, thyroid, prostate and isolated islet cells.

“GPCR activity” refers to the ability of a GPCR to transduce a signal. Such activity can be measured in a heterologous cell, by coupling a GPCR (or a chimeric GPCR) to either a G-protein or promiscuous G-protein such as Gα15, and an enzyme such as PLC, and measuring increases in intracellular calcium using well known techniques (Offermans & Simon, J. Biol. Chem. 270:15175-15180 (1995)). Receptor activity can be effectively measured by recording ligand-induced changes in [Ca²⁺]ᵢ using fluorescent Ca²⁺-indicator dyes and fluorometric imaging. Optionally, the polypeptides of the invention are involved in signal transduction, resulting in physiologic effects such increases or decreases of neurotransmitter or hormone release including insulin release, glucagon release, somatostatin release, pancreatic polypeptide release, VIP release, or serotonin release.

The phrase “functional effects” in the context of assays for testing compounds that modulate IC-GPCR mediated signal transduction includes the determination of any parameter that is indirectly or directly under the influence of the receptor, e.g., functional, physical and chemical effects. It includes ligand binding, changes in ion flux, membrane potential, current flow, transcription, G-protein binding, GPCR phosphorylation or dephosphorylation, signal transduction, receptor-ligand interactions, second messenger concentrations (e.g. cAMP, IP3, or intracellular Ca²⁺), in vitro, in vivo, and ex vivo and also includes other physiologic effects such increases or decreases of neurotransmitter or hormone release including insulin release, glucagon release, somatostatin release, pancreatic polypeptide release, VIP release, or serotonin release.

By “determining the functional effect” is meant assays for a compound that increases or decreases a parameter that is indirectly or directly under the influence of IC-GPCR, e.g., functional, physical and chemical effects. Such functional effects can be measured by any means known to those skilled in the art, e.g., changes in spectroscopic characteristics (e.g., fluorescence, absorbance, refractive index), hydrodynamic (e.g., shape), chromatographic, or solubility properties, patch clamping, voltage-sensitive dyes, whole cell currents, radioisotope efflux, inducible markers, oocyte IC-GPCR expression; tissue culture cell IC-GPCR expression; transcriptional activation of IC-GPCR; ligand binding assays; voltage, membrane potential and conductance changes; ion flux assays; changes in intracellular second messengers such as cAMP and inositol triphosphate (IP3); changes in intracellular calcium levels; neurotransmitter release, insulin release, glucagon release, somatostatin release, pancreatic polypeptide release, VIP release, serotonin release, and the like.

“Inhibitors,” “activators,” and “modulators” of IC-GPCR are used interchangeably to refer to inhibitory, activating, or modulating molecules identified using in vitro and in vivo assays for islet cell signal transduction, e.g., ligands, agonists, antagonists, and their homologs and mimetics. Inhibitors are compounds that, e.g., bind to, partially or totally block stimulation, decrease, prevent, delay activation, inactivate, desensitize, or down regulate signal transduction, e.g., antagonists. Activators are compounds that, e.g., bind to, stimulate, increase, open, activate, facilitate, enhance activation, sensitize or up regulate islet cell signal transduction, e.g., agonists. Modulators include compounds that, e.g., alter the interaction of a receptor with: extracellular proteins that bind activators or inhibitor (e.g., ebnerin and other members of the hydrophobic carrier family); G-proteins; kinases (e.g., homologs of rhodopsin kinase and beta adrenergic receptor kinases that are involved in deactivation and desensitization of a receptor); and arrestin-like proteins, which also deactivate and desensitize receptors. Modulators include genetically modified versions of IC-GPCR, e.g., with altered activity, as well as naturally occurring and synthetic ligands, antagonists, agonists, small chemical molecules and the like. Such assays for inhibitors and activators include, e.g., expressing IC-GPCR in cells or cell membranes, applying putative modulator compounds, and then determining the functional effects on signal transduction, as described above. Samples or assays comprising IC-GPCR that are treated with a potential activator, inhibitor, or modulator are compared to control samples without the inhibitor, activator, or modulator to examine the extent of inhibition. Control samples (untreated with inhibitors) are assigned a relative IC-GPCR activity value of 100%. Inhibition of IC-GPCR is achieved when the IC-GPCR activity value relative to the control is about 80%, optionally 50% or 25-0%. Activation of IC-GPCR is achieved when the IC-GPCR activity value relative to the control is 110%, optionally 150%, optionally 200-500%, or 1000-3000% higher.

“Biologically active” IC-GPCR refers to IC-GPCR having GPCR activity as described above, involved in islet cell signal transduction.

The terms “isolated” “purified” or “biologically pure” refer to material that is substantially or essentially free from components which normally accompany it as found in its native state. Purity and homogeneity are typically determined using analytical chemistry techniques such as polyacrylamide gel electrophoresis or high performance liquid chromatography. A protein that is the predominant species present in a preparation is substantially purified. In particular, an isolated IC-GPCR nucleic acid is separated from open reading frames that flank the IC-GPCR gene and encode proteins other than IC-GPCR. The term “purified” denotes that a nucleic acid or protein gives rise to essentially one band in an electrophoretic gel. Particularly, it means that the nucleic acid or protein is at least 85% pure, optionally at least 95% pure, and optionally at least 99% pure.

“Nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanihe is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:

1) Alanine (A), Glycine (G);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cysteine (C), Methionine (M)

(see, e.g., Creighton, Proteins (1984)).

Macromolecular structures such as polypeptide structures can be described in terms of various levels of organization. For a general discussion of this organization, see, e.g., Alberts et al., Molecular Biology of the Cell (3^{rd} ed., 1994) and Cantor and Schimmel, Biophysical Chemistry Part I: The Conformation of Biological Macromolecules (1980). “Primary structure” refers to the amino acid sequence of a particular peptide. “Secondary structure” refers to locally ordered, three dimensional structures within a polypeptide. These structures are commonly known as domains. Domains are portions of a polypeptide that form a compact unit of the polypeptide and are typically 50 to 350 amino acids long. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed by the noncovalent association of independent tertiary units. Anisotropic terms are also known as energy terms.

A “label” or a “detectable moiety” is a composition detectable by spectroscopic, photochemical, biochemical, immunochemical, or chemical means. For example, useful labels include ³²P, fluorescent dyes, electron-dense reagents, enzymes (e.g., as commonly used in an ELISA), biotin, digoxigenin, or haptens and proteins for which antibodies can be made detectable, e.g., by incorporating a radiolabel into the peptide, and used to detect antibodies specifically reactive with the peptide).

A “labeled nucleic acid probe or oligonucleotide” is one that is bound, either covalently, through a linker or a chemical bond, or noncovalently, through ionic, van der Waals, electrostatic, or hydrogen bonds to a label such that the presence of the probe may be detected by detecting the presence of the label bound to the probe.

As used herein a “nucleic acid probe or oligonucleotide” is defined as a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (7-deazaguanosine, inosine, etc.). In addition, the bases in a probe may be joined by a linkage other than a phosphodiester bond, so long as it does not interfere with hybridization. Thus, for example, probes may be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions. The probes are optionally directly labeled as with isotopes, chromophores, lumiphores, chromogens, or indirectly labeled such as with biotin to which a streptavidin complex may later bind. By assaying for the presence or absence of the probe, one can detect the presence or absence of the select sequence or subsequence.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or-express native genes that are otherwise abnormally expressed, under expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

The terms “identical” or percent “identity,” in the context of two or more nucleic acids or polypeptide sequences, refer to two or more sequences or subsequences that are the same or have a specified percentage of amino acid residues or nucleotides that are the same (i.e., 70% identity, optionally 75%, 80%, 85%, 90%, or 95% identity over a specified region), when compared and aligned for maximum correspondence over a comparison window, or designated region as measured using one of the following sequence comparison algorithms or by manual alignment and visual inspection. Such sequences are then said to be “substantially identical.” This definition also refers to the compliment of a test sequence. Optionally, the identity exists over a region that is at least about 50 amino acids or nucleotides in length, or more preferably over a region that is 75-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

One example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive aliginment method of Feng & Doolittle, J. Mol. Evol. 35:351-360 (1987). The method used is similar to the method described by Higgins & Sharp, CABIOS 5:151-153 (1989). The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al., Nuc. Acids Res. 12:387-395 (1984).

Another example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The phrase “selectively (or specifically) hybridizes to” refers to the binding, duplexing, or hybridizing of a molecule only to a particular nucleotide sequence under stringent hybridization conditions when that sequence is present in a complex mixture (eg., total cellular or library DNA or RNA).

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic acid concentration) at which 50% oaf the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, optionally 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′_{2,} a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

For preparation of monoclonal or polyclonal antibodies, any technique known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy (1985)). Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

An “anti-IC-GPCR” antibody is an antibody or antibody fragment that specifically binds a polypeptide encoded by the IC-GPCR gene, cDNA, or a subsequence thereof.

The term “immunoassay” is an assay that uses an antibody to specifically bind an antigen. The immunoassay is characterized by the use of specific binding properties of a particular antibody to isolate, target, and/or quantify the antigen.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised to IC-GPCR from specific species such as rat, murine, or human can be selected to obtain only those polyclonal antibodies that are specifically immunoreactive with IC-GPCR and not with other proteins, except for polymorphic variants and alleles of IC-GPCR. This selection may be achieved by subtracting out antibodies that cross-react with IC-GPCR molecules from other species. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

The phrase “selectively associates with” refers to the ability of a nucleic acid to “selectively hybridize” with another as defined above, or the ability of an antibody to “selectively (or specifically) bind to a protein, as defined above.

By “host cell” is meant a cell that contains an expression vector and supports the replication or expression of the expression vector. Host cells may be prokaryotic cells such as E. coli, or eukaryotic cells such as yeast, insect, amphibian, or mammalian cells such as CHO, HeLa and the like, e.g., cultured cells, explants, and cells in vivo.

### BRIEF DESCRIPTION OF THE DRAWINGS

FIG. 1 illustrates the expression pattern of probe set MBXMUSISL23905 in a murine pancreatic β cell line, murine islets and various murine tissues.

FIG. 2 illustrates the alignment of the murine IC-GPCR amino acid sequence with other members of the GPCR superfamily of proteins.

FIG. 3 illustrates the transmembrane domains predicted in the human and murine IC-GPCR sequences. The 7 transmembrane domains for mouse and human ISR were predicted by SMART analysis (Simple Modular Architecture Research Tool, http://smart.embl-heidelberg.de/). Similar results were obtained when using different prediction programs, including the Tmpred program (BCM search launcher: protein secondary structure prediction—http://dot.imgen.bcm.tmc.edu:9331/seq-search/struc-predict.html).

FIG. 4 illustrates the alignment of the murine and human IC-GPCR amino acid sequences.

FIG. 5 illustrates the expression pattern of the human IC-GPCR gene in various human tissues.

FIG. 6 illustrates the expression levels of murine IC-GPCR in wild-type and diabetic islets.

FIG. 7 illustrates the binding of an affinity-purified antibody directed to the C-terminal peptide of IC-GPCR to recombinant IC-GPCR protein expressed in HEK293T fibroblasts transfected with the human IC-GPCR cDNA in pcDNA12.2 (Invitrogen (Gateway)) but not in control cells transfected with a lacZ cDNA in the same vector. A: Western blot shows a prominent band at the size expected for IC-GPCR in pcDNA12.2-IC-GPCR transfected cells, but nothing is detected in pcDNA12.2-lacZ transfected cells. B: Immunofluorescent detection of IC-GPCR in pcDNA12.2-IC-GPCR transfected HEK293T fibroblasts demonstrates plasma membrane localization of the recombinant protein. There was no significant fluorescence observed in pcDNA12.2-lacZ transfected cells.

FIG. 8 illustrates the immunofluorescent detection of human IC-GPCR in human pancreatic islets. Sections of human pancreatic tissue were stained with the same affinity-purified antibody used in FIG. 7. IC-GPCR is readily detected in the islets but is not detected in the surrounding acinar tissue.

FIG. 9 illustrates the immunofluorescent co-localization of mouse IC-GPCR and insulin in mouse pancreatic islets from chow fed and 58% fat fed C57BL/6J mice. IC-GPCR is abundant in insulin positive cells in both diets and absent from the surrounding acinar tissue. The increased fluorescence observed in high fat fed mice presumably indicates increased abundance of the protein. This phenomenon is observed in the vast majority of islets of fat fed mice relative to chow fed mice in both C57BL/6J and DBA strains.

### DETAILED DESCRIPTION

### I. Introduction

The present invention provides for the first time nucleic acids encoding an islet cell G-protein coupled receptor. These nucleic acids and the receptors that they encode are referred to as as “IC-GPCR” for islet cell G-protein coupled receptors. The islet cell GPCR are components of the signal transduction pathway in islet cells in the pancreas. These nucleic acids provide valuable probes for the identification of islet cells, as the nucleic acids are specifically expressed in islet cells. Furthermore, the nucleic acids and the proteins they encode can be used as probes to dissect signal transduction in the islet cells.

The invention also provides methods of screening for modulators, e.g., activators, inhibitors, stimulators, enhancers, agonists, and antagonists, of these novel islet cell GPCRs. Such modulators of signal transduction are useful for pharmacological and genetic modulation of islet cell signaling pathways and for treating diabetes. These methods of screening can be used to identify high affinity agonists and antagonists of islet cell activity. These modulatory compounds can then be used to alter islet cell response to external stimuli. Thus, the invention provides assays for islet cell signal modulation, where IC-GPCR acts as a direct or indirect reporter molecule for the effect of modulators on islet cell signal transduction and insulin release, glucagon release, somatostatin release, pancreatic polypeptide release, VIP release, serotonin release, and the like. IC-GPCRs can be used in assays, e.g., to measure changes in ion concentration, membrane potential, current flow, ion flux, transcription, signal transduction, receptor-ligand interactions, second messenger concentrations, in vitro, in vivo, and ex vivo. In one embodiment, IC-GPCR can be used as an indirect reporter via attachment to a second reporter molecule such as green fluorescent protein (see, e.g., Mistili & Spector, Nature Biotechnology 15:961-964 (1997)). In another embodiment, IC-GPCR is recombinantly expressed in cells, and modulation of signal transduction via GPCR activity is assayed by measuring changes in Ca²⁺ levels.

Methods of assaying for modulators of islet cell signal transduction include in vitro ligand binding assays using IC-GPCR, portions thereof such as the extracellular domain, or chimeric proteins comprising one or more domains of IC-GPCR, oocyte IC-GPCR expression; tissue culture cell IC-GPCR expression; transcriptional activation of IC-GPCR; phosphorylation and dephosphorylation of GPCRs; G-protein binding to GPCRs; ligand binding assays; voltage, membrane potential and conductance changes; ion flux assays; changes in intracellular second messengers such as cAMP and inositol triphosphate; changes in intracellular calcium levels; changes in insulin release, glucagon release, somatostatin release, pancreatic polypeptide release, VIP release, serotonin release, neurotransmitter release and changes in islet cell proliferation, growth and/or death.

Finally, the invention provides for methods of detecting IC-GPCR nucleic acid and protein expression, allowing investigation of islet cell signal transduction, specific identification of islet cells, and diagnosing diabetes. IC-GPCR is useful as a nucleic acid probe for identifying islet cells. IC-GPCR can also be used to generate monoclonal and polyclonal antibodies also useful for identifying islet cells. Islet cells can be identified using techniques such as reverse transcription and amplification of mRNA, isolation of total RNA or poly A⁺ RNA, northern blotting, dot blotting, in situ hybridization, RNase protection, S1 digestion, probing DNA microchip arrays, western blots, and the like. Altered levels of IC-GPCR protein or nucleic acid correlates with the diabetic disease state.

Functionally, IC-GPCR represents a seven transmembrane G-protein coupled receptor involved in islet cell signal transduction, which interacts with a G-protein to mediate islet cell signal transduction (see, e.g., Fong, Cell Signal 8:217 (1996); Baldwin, Curr. Opin. Cell Biol. 6:180 (1994)).

Structurally, the nucleotide sequence of IC-GPCR (see, e.g., SEQ ID NOS:3 and 4, isolated from human and murine respectively) encodes a polypeptide of approximately 374 and 348 amino acids for the human and murine proteins respectively with a predicted molecular weights of approximately 40 kDa and 38 kDa respectively (see, e.g., SEQ ID NOS:1 and 2, isolated from human and murine respectively). Related IC-GPCR genes from other species share at least about 70% amino acid identity over a amino acid region at least about 25 amino acids in length, optionally 50 to 100 amino acids in length. IC-GPCR is specifically expressed pancreatic islet cells.

Specific regions of the IC-GPCR nucleotide and amino acid sequence may be used to identify polymorphic variants, interspecies homologs, and alleles of IC-GPCR. This identification can be made in vitro, e.g., under stringent hybridization conditions or PCR (using primers amplifying conserved regions of IC-GPCR) and sequencing, or by using the sequence information in a computer system for comparison with other nucleotide sequences. Typically, identification of polymorphic variants and alleles of IC-GPCR is made by comparing an amino acid sequence of about 25 amino acids or more, e.g., 50-100 amino acids. Amino acid identity of approximately at least 70% or above, optionally 80% or 90-95% or above typically demonstrates that a protein is a polymorphic variant, interspecies homolog, or allele of IC-GPCR. Sequence comparison can be performed using any of the sequence comparison algorithms discussed below. Antibodies that bind specifically to IC-GPCR or a conserved region thereof can also be used to identify alleles, interspecies homologs, and polymorphic variants.

Polymorphic variants, interspecies homologs, and alleles of IC-GPCR are confirmed by examining islet cell specific expression of the putative IC-GPCR polypeptide. Typically, IC-GPCR having the amino acid sequence of SEQ ID NO:1 or 2 is used as a positive control in comparison to the putative IC-GPCR protein to demonstrate the identification of a polymorphic variant or allele of IC-GPCR. The polymorphic variants, alleles and interspecies homologs are expected to retain the seven transmembrane structure of a G-protein coupled receptor.

IC-GPCR nucleotide and amino acid sequence information may also be used to construct models of IC-GPCR polypeptides in a computer system. These models are subsequently used to identify compounds that can activate or inhibit IC-GPCR. Such compounds that modulate the activity of IC-GPCR can be used to investigate the role of IC-GPCR in islet cell signal transduction and can be used to treat diabetes.

The isolation of IC-GPCR for the first time provides a means for assaying for inhibitors and activators of G-protein coupled receptor islet cell signal transduction. Biologically active IC-GPCR is useful for testing inhibitors and activators of IC-GPCR as signal transducers using in vivo and in vitro expression that measure, e.g., transcriptional activation of IC-GPCR; ligand binding; phosphorylation and dephosphorylation; binding to G-proteins; G-protein activation; regulatory molecule binding; voltage, membrane potential and conductance changes; ion flux; intracellular second messengers such as cAMP and inositol triphosphate; intracellular calcium levels; insulin release, glucagon release, somatostatin release, pancreatic polypeptide release, VIP release, serotonin release, and neurotransmitter release. Such activators and inhibitors identified using IC-GPCR, can be used to further study islet cell signal transduction and to identify specific agonists and antagonists. Such activators and inhibitors are useful as pharmaceutical agents for modulating islet cell activity including modulating insulin release, glucagon release, somatostatin release, pancreatic polypeptide release, VIP release, serotonin release, and the like, treating diabetes and for modulating islet cell proliferation, growth and/or death.

Methods of detecting IC-GPCR nucleic acids and expression of IC-GPCR are also useful for identifying normal and diseased islet cells expressing IC-GPCR. Chromosome localization of the genes encoding murine and human IC-GPCR related sequences can be used to identify diseases, mutations, and traits caused by and associated with related IC-GPCR genes.

### II. Isolation of the Nucleic Acid Encoding IC-GPCR

A. General Recombinant DNA Methods

This invention relies on routine techniques in the field of recombinant genetics. Basic texts disclosing the general methods of use in this invention include Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994)).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized according to the solid phase phosphoramidite triester method first described by Beaucage & Caruthers, Tetrahedron Letts. 22:1859-1862 (1981), using an automated synthesizer, as described in Van Devanter et. al., Nucleic Acids Res. 12:6159-6168 (1984). Purification of oligonucleotides is by either native acrylamide gel electrophoresis or by anion-exchange HPLC as described in Pearson & Reanier, J. Chrom. 255:137-149 (1983).

The sequence of the cloned genes and synthetic oligonucleotides can be verified after cloning using, e.g., the chain termination method for sequencing double-stranded templates of Wallace et al., Gene 16:21-26 (1981).

B. Cloning Methods for the Isolation of Nucleotide Sequences Encoding IC-GPCR

In general, the nucleic acid sequences encoding IC-GPCR and related nucleic acid sequence homologs are cloned from cDNA and genomic DNA libraries by hybridization with a probe, or isolated using amplification techniques with oligonucleotide primers. For example, IC-GPCR sequences are typically isolated from mammalian nucleic acid (genomic or cDNA) libraries by hybridizing with a nucleic acid probe, the sequence of which can be derived from SEQ ID NOs:3 and 4. A suitable tissue from which IC-GPCR RNA and cDNA can be isolated is pancreatic tissue, optionally individual islet cells.

Amplification techniques using primers can also be used to amplify and isolate IC-GPCR from DNA or RNA. The primers identified in SEQ ID NO:5 and 6 can be used to amplify a sequence of human IC-GPCR: and the primers identified in SEQ ID NO:7 and 8 can be used to amplify a sequence of mouse IC-GPCR (see, e.g., Dieffenfach & Dveksler, PCR Primer: A Laboratory Manual (1995)). Primers derived from SEQ ID NO:3 and 4 can be used, e.g., to amplify either the full length sequence or a probe of one to several hundred nucleotides, which is then used to screen a mammalian library for full-length human or mouse IC-GPCR respectively.

Nucleic acids encoding IC-GPCR can also be isolated from expression libraries using antibodies as probes. Such polyclonal or monoclonal antibodies can be raised using the sequence of SEQ ID NOs:1 and 2.

IC-GPCR polymorphic variants, alleles, and interspecies homologs that are substantially identical to IC-GPCR can be isolated using IC-GPCR nucleic acid probes, and oligonucleotides under stringent hybridization conditions, by screening libraries. Alternatively, expression libraries can be used to clone IC-GPCR and IC-GPCR polymorphic variants, alleles, and interspecies homologs, by detecting expressed homologs immunologically with antisera or purified antibodies made against IC-GPCR, which also recognize and selectively bind to the IC-GPCR homolog.

To make a cDNA library, one should choose a source that is rich in IC-GPCR mRNA, e.g., pancreatic tissue, isolated islets, or isolated islet cells. The mRNA is then made into cDNA using reverse transcriptase, ligated into a recombinant vector, and transfected into a recombinant host for propagation, screening and cloning. Methods for making and screening cDNA libraries are well known (see, e.g., Gubler & Hoffman, Gene 25:263-269 (1983); Sambrook et al., supra; Ausubel et al., supra).

For a genomic library, the DNA is extracted from the tissue and either mechanically sheared or enzymatically digested to yield fragments of about 12-20 kb. The fragments are then separated by gradient centrifugation from undesired sizes and are constructed in bacteriophage lambda vectors. These vectors and phage are packaged in vitro. Recombinant phage are analyzed by plaque hybridization as described in Benton & Davis, Science 196:180-182 (1977). Colony hybridization is carried out as generally described in Grunstein et al., Proc. Natl. Acad. Sci. USA., 72:3961-3965 (1975).

An alternative method of isolating IC-GPCR nucleic acid and its homologs combines the use of synthetic oligonucleotide primers and amplification of an RNA or DNA template (see U.S. Pat. Nos. 4,683,195 and 4,683,202; PCR Protocols: A Guide to Methods and Applications (Innis et al., eds, 1990)). Methods such as polymerase chain reaction (PCR) and ligase chain reaction (LCR) can be used to amplify nucleic acid sequences of IC-GPCR directly from mRNA, from cDNA, from genomic libraries or cDNA libraries. Degenerate oligonucleotides can be designed to amplify IC-GPCR homologs using the sequences provided herein. Restriction endonuclease sites can be incorporated into the primers. Polymerase chain reaction or other in vitro amplification methods may also be useful, for example, to clone nucleic acid sequences that code for proteins to be expressed, to make nucleic acids to use as probes for detecting the presence of IC-GPCR encoding mRNA in physiological samples, for nucleic acid sequencing, or for other purposes. Genes amplified by the PCR reaction can be purified from agarose gels and cloned into an appropriate vector.

Gene expression of IC-GPCR can also be analyzed by techniques known in the art, e.g., reverse transcription and amplification of mRNA, isolation of total RNA or poly A⁺ RNA, northern blotting, dot blotting, in situ hybridization, RNase protection, probing DNA microchip arrays, and the like. In one embodiment, high density oligonucleotide analysis technology (e.g., GENECHIP®) is used to identify homologs and polymorphic variants of the IC-GPCRs of the invention. In the case where the homologs being identified are linked to a known disease, they can be used with GENECHIP® as a diagnostic tool in detecting the disease in a biological sample, see, e.g., Gunthand et al., AIDS Res. Hum. Retroviruses 14: 869-876 (1998); Kozal et al., Nat. Med. 2:753-759 (1996); Matson et al., Anal. Biochem. 224:110-106 (1995); Lockhart et al., Nat. Biotechnol. 14:1675-1680 (1996); Gingeras et al., Genome Res. 8:435-448 (1998); Hacia et al., Nucleic Acids Res. 26:3865-3866 (1998).

Synthetic oligonucleotides can be used to construct recombinant IC-GPCR genes for use as probes or for expression of protein. This method is performed using a series of overlapping oligonucleotides usually 40-120 bp in length, representing both the sense and non-sense strands of the gene. These DNA fragments are then annealed, ligated and cloned. Alternatively, amplification techniques can be used with precise primers to amplify a specific subsequence of the IC-GPCR nucleic acid. The specific subsequence is then ligated into an expression vector.

The nucleic acid encoding IC-GPCR is typically cloned into intermediate vectors before transformation into prokaryotic or eukaryotic cells for replication and/or expression. These intermediate vectors are typically prokaryote vectors, e.g., plasmids, or shuttle vectors.

Optionally, nucleic acids encoding chimeric proteins comprising IC-GPCR or domains thereof can be made according to standard techniques. For example, a domain such as ligand binding domain, an extracellular domain, a transmembrane domain (e.g., one comprising seven transmembrane regions and corresponding extracellular and cytosolic loops), the transmembrane domain and a cytoplasmic domain, an active site, a subunit association region, etc., can be covalently linked to a heterologous protein. For example, an extracellular domain can be linked to a heterologous GPCR transmembrane domain, or a heterologous GPCR extracellular domain can be linked to a transmembrane domain. Other heterologous proteins of choice include, e.g., green fluorescent protein, β-gal, glutamate receptor, and the rhodopsin presequence.

C. Expression in Prokaryotes and Eukaryotes

To obtain high level expression of a cloned gene or nucleic acid, such as those cDNAs encoding IC-GPCR, one typically subclones IC-GPCR into an expression vector that contains a strong promoter to direct transcription, a transcription/translation terminator, and if for a nucleic acid encoding a protein, a ribosome binding site for translational initiation. Suitable bacterial promoters are well known in the art and described, e.g., in Sambrook et al. supra and Ausubel et al. supra. Bacterial expression systems for expressing the IC-GPCR protein are available in, e.g., E. coli, Bacillus sp., and Salmonella (Palva et al., Gene 22:229-235 (1983); Mosbach et al., Nature 302:543-545 (1983). Kits for such expression systems are commercially available. Eukaryotic expression systems for mammalian cells, yeast, and insect cells are well known in the art and are also commercially available. In one embodiment, the eukaryotic expression vector is an adenoviral vector, an adeno-associated vector, or a retroviral vector.

The promoter used to direct expression of a heterologous nucleic acid depends on the particular application. The promoter is optionally positioned about the same distance from the heterologous transcription start site as it is from the transcription start site in its natural setting. As is known in the art, however, some variation in this distance can be accommodated without loss of promoter function.

In addition to the promoter, the expression vector typically contains a transcription unit or expression cassette that contains all the additional elements required for the expression of the IC-GPCR encoding nucleic acid in host cells. A typical expression cassette thus contains a promoter operably linked to the nucleic acid sequence encoding IC-GPCR and signals required for efficient polyadenylation of the transcript, ribosome binding sites, and translation termination. The nucleic acid sequence encoding IC-GPCR may typically be linked to a cleavable signal peptide sequence to promote secretion of the encoded protein by the transformed cell. Such signal peptides would include, among others, the signal peptides from tissue plasminogen activator, insulin, and neuron growth factor, and juvenile hormone esterase of Heliothis virescens. Additional elements of the cassette may include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as GST and LacZ. Epitope tags can also be added to recombinant proteins to provide convenient methods of isolation, e.g., c-myc.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A⁺, pMTO10/A⁺, pMAMneo-5, baculovirus pDSVE, and any other vector allowing expression of proteins under the direction of the SV40 early promoter, SV40 later promoter, metallothionein promoter, murine mammary tumor virus promoter, Rous sarcoma virus promoter, polyhedrin promoter, or other promoters shown effective for expression in eukaryotic cells.

Some expression systems have markers that provide gene amplification such as thymidine kinase, hygromycin B phosphotransferase, and dihydrofolate reductase. Alternatively, high yield expression systems not involving gene amplification are also suitable, such as using a baculovirus vector in insect cells, with a IC-GPCR encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are optionally chosen such that they do not interfere with the replication of the DNA in eukaryotic cells, if necessary.

Standard transfection methods are used to produce bacterial, mammalian, yeast or insect cell lines that express large quantities of IC-GPCR protein, which are then purified using standard techniques (see, e.g., Colley et al., J. Biol. Chem. 264:17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132:349-351 (1977); Clark-Curtiss & Curtiss, Methods in Enzymology 101:347-362 (Wu et al., eds, 1983).

Any of the well known procedures for introducing foreign nucleotide sequences into host cells may be used. These include the use of calcium phosphate transfection, polybrene, protoplast fusion, electroporation, liposomes, microinjection, plasma vectors, viral vectors and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing IC-GPCR.

After the expression vector is introduced into the cells, the transfected cells are cultured under conditions favoring expression of IC-GPCR, which is recovered from the culture using standard techniques identified below.

### III. Purification of IC-GPCR

Either naturally occurring or recombinant IC-GPCR can be purified for use in functional assays. Naturally occurring IC-GPCR is purified, e.g., from mammalian tissue such as pancreatic islet cell tissue, and any other source of a IC-GPCR homolog. Recombinant IC-GPCR is purified from any suitable bacterial or eukaryotic expression system, e.g., CHO cells or insect cells.

IC-GPCR may be purified to substantial purity by standard techniques, including selective precipitation with such substances as ammonium sulfate; column chromatography, immunopurification methods, and others (see, e.g., Scopes, Protein Purification: Principles and Practice (1982); U.S. Pat. No. 4,673,641; Ausubel et al., supra; and Sambrook et al., supra).

A number of procedures can be employed when recombinant IC-GPCR is being purified. For example, proteins having established molecular adhesion properties can be reversible fused to IC-GPCR. With the appropriate ligand, IC-GPCR can be selectively adsorbed to a purification column and then freed from the column in a relatively pure form. The fused protein is then removed by enzymatic activity. Finally IC-GPCR could be purified using immunoaffinity columns.

A. Purification of IC-GPCR from Recombinant Cells

Recombinant proteins are expressed by transformed bacteria or eukaryotic cells such as CHO cells or insect cells in large amounts, typically after promoter induction; but expression can be constitutive. Promoter induction with IPTG is one example of an inducible promoter system. Cells are grown according to standard procedures in the art. Fresh or frozen cells are used for isolation of protein.

Proteins expressed in bacteria may form insoluble aggregates (“inclusion bodies”). Several protocols are suitable for purification of IC-GPCR inclusion bodies. For example, purification of inclusion bodies typically involves the extraction, separation and/or purification of inclusion bodies by disruption of bacterial cells, e.g., by incubation in a buffer of 50 mM TRIS/HCL pH 7.5, 50 mM NaCl, 5 mM MgCl₂, 1 mM DTT, 0.1 mM ATP, and 1 mM PMSF. The cell suspension can be lysed using 2-3 passages through a French Press, homogenized using a Polytron (Brinkman Instruments) or sonicated on ice. Alternate methods of lysing bacteria are apparent to those of skill in the art (see, e.g., Sambrook et al., supra; Ausubel et al., supra).

If necessary, the inclusion bodies are solubilized, and the lysed cell suspension is typically centrifuged to remove unwanted insoluble matter. Proteins that formed the inclusion bodies-may be renatured by dilution or dialysis with a compatible buffer. Suitable solvents include, but are not limited to urea (from about 4 M to about 8 M), formamide (at least about 80%, volume/volume basis), and guanidine hydrochloride (from about 4 M to about 8 M). Some solvents which are capable of solubilizing aggregate-forming proteins, for example SDS (sodium dodecyl sulfate), 70% formic acid, are inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing re-formation of immunologically and/or biologically active protein. Other suitable buffers are known to those skilled in the art. IC-GPCR is separated from other bacterial proteins by standard separation techniques, e.g., with Ni-NTA agarose resin.

Alternatively, it is possible to purify IC-GPCR from bacteria periplasm. After lysis of the bacteria, when IC-GPCR is exported into the periplasm of the bacteria, the periplasmic fraction of the bacteria can be isolated by cold osmotic shock in addition to other methods known to skill in the art. To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO₄ and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

B. Standard Protein Separation Techniques for Purifying IC-GPCR

1. Solubility Fractionation

Often as an initial step, particularly if the protein mixture is complex, an initial salt fractionation can separate many of the unwanted host cell proteins (or proteins derived from the cell culture media) from the recombinant protein of interest. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol includes adding saturated ammonium sulfate to a protein solution so that the resultant ammonium sulfate concentration is between 20-30%. This concentration will precipitate the most hydrophobic of proteins. The precipitate is then discarded (unless the protein of interest is hydrophobic) and ammonium sulfate is added to the supernatant to a concentration known to precipitate the protein of interest. The precipitate is then solubilized in buffer and the excess salt removed if necessary, either through dialysis or diafiltration. Other methods that rely on solubility of proteins, such as cold ethanol precipitation, are well known to those of skill in the art and can be used to fractionate complex protein mixtures.

2. Size Differential Filtration

The molecular weight of IC-GPCR can be used to isolated it from proteins of greater and lesser size using ultrafiltration through membranes of different pore size (for example, Amicon or Millipore membranes). As a first step, the protein mixture is ultrafiltered through a membrane with a pore size that has a lower molecular weight cut-off than the molecular weight of the protein of interest. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

3. Column Chromatography

IC-GPCR can also be separated from other proteins on the basis of its size, net surface charge, hydrophobicity, and affinity for ligands. In addition, antibodies raised against proteins can be conjugated to column matrices and the proteins immunopurified. All of these methods are well known in the art. It will be apparent to one of skill that chromatographic techniques can be performed at any scale and using equipment from many different manufacturers (e.g., Pharmacia Biotech).

### IV. Immunological Detection of IC-GPCR

In addition to the detection of IC-GPCR genes and gene expression using nucleic acid hybridization technology, one can also use immunoassays to detect IC-GPCR, e.g., to identify islet cells expressing IC-GPCR and variants of IC-GPCR. Immunoassays can be used to qualitatively or quantitatively analyze IC-GPCR. A general overview of the applicable technology can be found in Harlow & Lane, Antibodies: A Laboratory Manual (1988).

A. Antibodies to IC-GPCR

Methods of producing polyclonal and monoclonal antibodies that react specifically with IC-GPCR are known to those of skill in the art (see, e.g., Coligan, Current Protocols in Immunology (1991); Harlow & Lane, supra; Goding, Monoclonal Antibodies: Principles and Practice (2d ed. 1986); and Kohler & Milstein, Nature 256:495-497 (1975). Such techniques include antibody preparation by selection of antibodies from libraries of recombinant antibodies in phage or similar vectors, as well as preparation of polyclonal and monoclonal antibodies by immunizing rabbits or mice (see, e.g., Huse et al., Science 246:1275-1281 (1989); Ward et al., Nature 341:544-546 (1989)).

A number of IC-GPCR comprising immunogens may be used to produce antibodies specifically reactive with IC-GPCR. For example, recombinant IC-GPCR or an antigenic fragment thereof, is isolated as described herein. Recombinant protein can be expressed in eukaryotic or prokaryotic cells as described above, and purified as generally described above. Recombinant protein is the preferred immunogen for the production of monoclonal or polyclonal antibodies. Alternatively, a synthetic peptide derived from the sequences disclosed herein and conjugated to a carrier protein can be used an immunogen. Naturally occurring protein may also be used either in pure or impure form. The product is then injected into an animal capable of producing antibodies. Either monoclonal or polyclonal antibodies may be generated, for subsequent use in immunoassays to measure the protein.

Methods of production of polyclonal antibodies are known to those of skill in the art. An inbred strain of mice (e.g., BALB/C mice) or rabbits are immunized with the protein using a standard adjuvant, such as Freund's adjuvant, and a standard immunization protocol. The animal's immune response to the immunogen preparation is monitored by taking test bleeds and determining the titer of reactivity to IC-GPCR. When appropriately high titers of antibody to the immunogen are obtained, blood is collected from the animal and antisera are prepared. Further fractionation of the antisera to enrich for antibodies reactive to the protein can be done if desired (see Harlow & Lane, supra).

Monoclonal antibodies may be obtained by various techniques familiar to those skilled in the art. Briefly, spleen cells from an animal immunized with a desired antigen are immortalized, commonly by fusion with a myeloma cell (see Kohler & Milstein, Eur. J. Immunol. 6:511-519 (1976)). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according to the general protocol outlined by Huse et al., Science 246:1275-1281 (1989).

Monoclonal antibodies and polyclonal sera are collected and titered against the immunogen protein in an immunoassay, for example, a solid phase immunoassay with the immunogen immobilized on a solid support. Typically, polyclonal antisera with a titer of 10⁴ or greater are selected and tested for their cross reactivity against non-IC-GPCR proteins or even other related proteins from other organisms, using a competitive binding immunoassay. Specific polyclonal antisera and monoclonal antibodies will usually bind with a K_{d} of at least about 0.1 mM, more usually at least about 1 μM, optionally at least about 0.1 μM or better, and optionally 0.01 μM or better.

Once IC-GPCR specific antibodies are available, IC-GPCR can be detected by a variety of immunoassay methods. For a review of immunological and immunoassay procedures, see Basic and Clinical Immunology (Stites & Terr eds., 7th ed. 1991). Moreover, the immunoassays of the present invention can be performed in any of several configurations, which are reviewed extensively in Enzyme Immunoassay (Maggio, ed., 1980); and Harlow & Lane, supra.

B. Immunological Binding Assays

IC-GPCR can be detected and/or quantified using any of a number of well recognized immunological binding assays (see, e.g., U.S. Pat. Nos. 4,366,241; 4,376,110; 4,517,288; and 4,837,168). For a review of the general immunoassays, see also Methods in Cell Biology: Antibodies in Cell Biology, volume 37 (Asai, ed. 1993); Basic and Clinical Immunology (Stites & Terr, eds., 7th ed. 1991). Immunological binding assays (or immunoassays) typically use an antibody that specifically binds to a protein or antigen of choice (in this case the IC-GPCR or antigenic subsequence thereof). The antibody (e.g., anti-IC-GPCR) may be produced by any of a number of means well known to those of skill in the art and as described above.

Immunoassays also often use a labeling agent to specifically bind to and label the complex formed by the antibody and antigen. The labeling agent may itself be one of the moieties comprising the antibody/antigen complex. Thus, the labeling agent may be a labeled IC-GPCR polypeptide or a labeled anti-IC-GPCR antibody. Alternatively, the labeling agent may be a third moiety, such a secondary antibody, that specifically binds to the antibody/IC-GPCR complex (a secondary antibody is typically specific to antibodies of the species from which the first antibody is derived). Other proteins capable of specifically binding immunoglobulin constant regions, such as protein A or protein G may also be used as the label agent. These proteins exhibit a strong non-immunogenic reactivity with immunoglobulin constant regions from a variety of species (see, e.g., Kronval et al., J. Immunol. 111: 1401-1406 (1973); Akerstrom et al., J. Immunol. 135:2589-2589 (1985)). The labeling agent can be modified with a detectable moiety, such as biotin, to which another molecule can specifically bind, such as streptavidin. A variety of detectable moieties are well known to those skilled in the art.

Throughout the assays, incubation and/or washing steps may be required after each combination of reagents. Incubation steps can vary from about 5 seconds to several hours, optionally from about 5 minutes to about 24 hours. However, the incubation time will depend upon the assay format, antigen, volume of solution, concentrations, and the like. Usually, the assays will be carried out at ambient temperature, although they can be conducted over a range of temperatures, such as 10° C. to 40° C.

1. Non-Competitive Assay Formats

Immunoassays for detecting IC-GPCR in samples may be either competitive or noncompetitive. Noncompetitive immunoassays are assays in which the amount of antigen is directly measured. In one preferred “sandwich” assay, for example, the anti-IC-GPCR antibodies can be bound directly to a solid substrate on which they are immobilized. These immobilized antibodies then capture IC-GPCR present in the test sample. IC-GPCR is thus immobilized is then bound by a labeling agent, such as a second IC-GPCR antibody bearing a label. Alternatively, the second antibody may lack a label, but it may, in turn, be bound by a labeled third antibody specific to antibodies of the species from which the second antibody is derived. The second or third antibody is typically modified with a detectable moiety, such as biotin, to which another molecule specifically binds, e.g., streptavidin, to provide a detectable moiety.

2. Competitive Assay Formats

In competitive assays, the amount of IC-GPCR present in the sample is measured indirectly by measuring the amount of a known, added (exogenous) IC-GPCR displaced (competed away) from an anti-IC-GPCR antibody by the unknown IC-GPCR present in a sample. In one competitive assay, a known amount of IC-GPCR is added to a sample and the sample is then contacted with an antibody that specifically binds to IC-GPCR. The amount of exogenous IC-GPCR bound to the antibody is inversely proportional to the concentration of IC-GPCR present in the sample. In a particularly preferred embodiment, the antibody is immobilized on a solid substrate. The amount of IC-GPCR bound to the antibody may be determined either by measuring the amount of IC-GPCR present in a IC-GPCR/antibody complex, or alternatively by measuring the amount of remaining uncomplexed protein. The amount of IC-GPCR may be detected by providing a labeled IC-GPCR molecule.

A hapten inhibition assay is another preferred competitive assay. In this assay the known IC-GPCR, is immobilized on a solid substrate. A known amount of anti-IC-GPCR antibody is added to the sample, and the sample is then contacted with the immobilized IC-GPCR. The amount of anti-IC-GPCR antibody bound to the known immobilized IC-GPCR is inversely proportional to the amount of IC-GPCR present in the sample. Again, the amount of immobilized antibody may be detected by detecting either the immobilized fraction of antibody or the fraction of the antibody that remains in solution. Detection may be direct where the antibody is labeled or indirect by the subsequent addition of a labeled moiety that specifically binds to the antibody as described above.

3. Cross-Reactivity Determinations

Immunoassays in the competitive binding format can also be used for crossreactivity determinations. For example, a protein at least partially encoded by SEQ ID NOS:1 and 2 can be immobilized to a solid support. Proteins (e.g., IC-GPCR proteins and homologs) are added to the assay that compete for binding of the antisera to the immobilized antigen. The ability of the added proteins to compete for binding of the antisera to the immobilized protein is compared to the ability of IC-GPCR encoded by SEQ ID NO:1 or 2 to compete with itself. The percent crossreactivity for the above proteins is calculated, using standard calculations. Those antisera with less than 10% crossreactivity with each of the added proteins listed above are selected and pooled. The cross-reacting antibodies are optionally removed from the pooled antisera by immunoabsorption with the added considered proteins, e.g., distantly related homologs.

The immunoabsorbed and pooled antisera are then used in a competitive binding immunoassay as described above to compare a second protein, thought to be perhaps an allele or polymorphic variant of IC-GPCR, to the immunogen protein (i.e., IC-GPCR of SEQ ID NOS:1 or 2). In order to make this comparison, the two proteins are each assayed at a wide range of concentrations and the amount of each protein required to inhibit 50% of the binding of the antisera to the immobilized protein is determined. If the amount of the second protein required to inhibit 50% of binding is less than 10 times the amount of the protein encoded by SEQ ID NOS:1 or 2, that is required to inhibit 50% of binding, then the second protein is said to specifically bind to the polyclonal antibodies generated to a IC-GPCR immunogen.

4. Other Assay Formats

Western blot (immunoblot) analysis is used to detect and quantify the presence of IC-GPCR in the sample. The technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind IC-GPCR. The anti-IC-GPCR antibodies specifically bind to the IC-GPCR on the solid support. These antibodies may be directly labeled or alternatively may be subsequently detected using labeled antibodies (e.g., labeled sheep anti-murine antibodies) that specifically bind to the anti-IC-GPCR antibodies.

Other assay formats include liposome immunoassays (LIA), which use liposomes designed to bind specific molecules (e.g., antibodies) and release encapsulated reagents or markers. The released chemicals are then detected according to standard techniques (see Monroe et al., Amer. Clin. Prod. Rev. 5:34-41 (1986)).

5. Reduction of Non-Specific Binding

One of skill in the art will appreciate that it is often desirable to minimize non-specific binding in immunoassays. Particularly, where the assay involves an antigen or antibody immobilized on a solid substrate it is desirable to minimize the amount of non-specific binding to the substrate. Means of reducing such non-specific binding are well known to those of skill in the art. Typically, this technique involves coating the substrate with a proteinaceous composition. In particular, protein compositions such as bovine serum albumin (BSA), nonfat powdered milk, and gelatin are widely used with powdered milk being most preferred.

6. Labels

The particular label or detectable group used in the assay is not a critical aspect of the invention, as long as it does not significantly interfere with the specific binding of the antibody used in the assay. The detectable group can be any material having a detectable physical or chemical property. Such detectable labels have been well-developed in the field of immunoassays and, in general, most any label useful in such methods can be applied to the present invention. Thus, a label is any composition detectable by spectroscopic, photochemical, biochemical, immunochemical, electrical, optical or chemical means. Useful labels in the present invention include magnetic beads (e.g., DYNABEADS™), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic beads (e.g., polystyrene, polypropylene, latex, etc.).

The label may be coupled directly or indirectly to the desired component of the assay according to methods well known in the art. As indicated above, a wide variety of labels may be used, with the choice of label depending on sensitivity required, ease of conjugation with the compound, stability requirements, available instrumentation, and disposal provisions.

Non-radioactive labels are often attached by indirect means. Generally, a ligand molecule (e.g., biotin) is covalently bound to the molecule. The ligand then binds to another molecules (e.g., streptavidin) molecule, which is either inherently detectable or covalently bound to a signal system, such as a detectable enzyme, a fluorescent compound, or a chemiluminescent compound. The ligands and their targets can be used in any suitable combination with antibodies that recognize IC-GPCR, or secondary antibodies that recognize anti-IC-GPCR.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidotases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. For a review of various labeling or signal producing systems that may be used, see U.S. Pat. No. 4,391,904.

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Finally simple colorimetric labels may be detected simply by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. In this case, antigen-coated particles are agglutinated by samples comprising the target antibodies. In this format, none of the components need be labeled and the presence of the target antibody is detected by simple visual inspection.

### V. Assays for Modulators of IC-GPCR

A. Assays for IC-GPCR Activity

IC-GPCR and its alleles and polymorphic variants are G-protein coupled receptors that participate in islet cell signal transduction. The activity of IC-GPCR polypeptides can be assessed using a variety of in vitro and in vivo assays to determine functional, chemical, and physical effects, e.g., measuring ligand binding (e.g., radioactive ligand binding), second messengers (e.g., cAMP, cGMP, IP₃, DAG, or Ca²⁺), ion flux, phosphorylation levels, transcription levels, neurotransmitter levels, and the like. Furthermore, such assays can be used to test for inhibitors and activators of IC-GPCR. Modulators can also be genetically altered versions of IC-GPCR. Such modulators of islet cell signal transduction activity are useful for controlling aberrant islet cell signaling and treating diabetes.

The IC-GPCR of the assay will be selected from a polypeptide having a sequence of SEQ ID NOS:1 or 2 or conservatively modified variant thereof. Alternatively, the IC-GPCR of the assay will be derived from a eukaryote and include an amino acid subsequence having amino acid sequence identity SEQ ID NOS:1 or 2. Generally, the amino acid sequence identity will be at least 70%, optionally at least 85%, optionally at least 90-95%. Optionally, the polypeptide of the assays will comprise a domain of IC-GPCR, such as an extracellular domain, transmembrane domain, cytoplasmic domain, ligand binding domain, subunit association domain, active site, and the like. Either IC-GPCR or a domain thereof can be covalently linked to a heterologous protein to create a chimeric protein used in the assays described herein.

Modulators of IC-GPCR activity are tested using IC-GPCR polypeptides as described above, either recombinant or naturally occurring. The protein can be isolated, expressed in a cell, expressed in a membrane derived from a cell, expressed in tissue or in an animal, either recombinant or naturally occurring. For example, pancreatic slices, dissociated islet cells from a pancreas, transformed cells, or membranes can be used. Modulation is tested using one of the in vitro or in vivo assays described herein. Signal transduction can also be examined in vitro with soluble or solid state reactions, using a chimeric molecule such as an extracellular domain of a receptor covalently linked to a heterologous signal transduction domain, or a heterologous extracellular domain covalently linked to the transmembrane and or cytoplasmic domain of a receptor. Furthermore, ligand-binding domains of the protein of interest can be used in vitro in soluble or solid state reactions to assay for ligand binding.

Ligand binding to IC-GPCR, a domain, or chimeric protein can be tested in solution, in a bilayer membrane, attached to a solid phase, in a lipid monolayer, or in vesicles. Binding of a modulator can be tested using, e.g., changes in spectroscopic characteristics (e.g., fluorescence, absorbance, refractive index) hydrodynamic (e.g., shape), chromatographic, or solubility properties.

Receptor-G-protein interactions can also be examined. For example, binding of the G-protein to the receptor or its release from the receptor can be examined. For example, in the absence of GTP, an activator will lead to the formation of a tight complex of a G protein (all three subunits) with the receptor. This complex can be detected in a variety of ways, as noted above. Such an assay can be modified to search for inhibitors. Add an activator to the receptor and G protein in the absence of GTP, form a tight complex, and then screen for inhibitors by looking at dissociation of the receptor-G protein complex. In the presence of GTP, release of the alpha subunit of the G protein from the other two G protein subunits serves as a criterion of activation.

An activated or inhibited G-protein will in turn alter the properties of target enzymes, channels, and other effector proteins. The classic examples are the activation of cGMP phosphodiesterase by transducin in the visual system, adenylate cyclase by the stimulatory G-protein, phospholipase C by Gq and other cognate G proteins, and modulation of diverse channels by Gi and other G proteins. Downstream consequences can also be examined such as generation of diacyl glycerol and IP3 by phospholipase C, and in turn, for calcium mobilization by IP3.

Activated GPCR receptors become substrates for kinases that phosphorylate the C-terminal tail of the receptor (and possibly other sites as well). Thus, activators will promote the transfer of ³²P from gamma-labeled GTP to the receptor, which can be assayed with a scintillation counter. The phosphorylation of the C-terminal tail will promote the binding of arrestin-like proteins and will interfere with the binding of G-proteins. The kinase/arrestin pathway plays a key role in the desensitization of many GPCR receptors. For example, compounds that modulate the duration a of islet cell signal transduction would be useful as a means of treating diabetes. For a general review of GPCR signal transduction and methods of assaying signal transduction, see, e.g., Methods in Enzymology, vols. 237 and 238 (1994) and volume 96 (1983); Bourne et al., Nature 10:349:117-27 (1991); Bourne et al., Nature 348:125-32 (1990); Pitcher et al., Annu. Rev. Biochem. 67:653-92 (1998).

Samples or assays that are treated with a potential IC-GPCR inhibitor or activator are compared to control samples without the test compound, to examine the extent of modulation. Control samples (untreated with activators or inhibitors) are assigned a relative IC-GPCR activity value of 100. Inhibition of IC-GPCR is achieved when the IC-GPCR activity value relative to the control is about 90%, optionally 50%, optionally 25-0%. Activation of IC-GPCR is achieved when the IC-GPCR activity value relative to the control is 110%, optionally 150%, 200-500%, or 1000-2000%.

Changes in ion flux may be assessed by determining changes in polarization (i.e., electrical potential) of the cell or membrane expressing IC-GPCR. One means to determine changes in cellular polarization is by measuring changes in current (thereby measuring changes in polarization) with voltage-clamp and patch-clamp techniques, e.g., the “cell-attached” mode, the “inside-out” mode, and the “whole cell” mode (see, e.g., Ackerman et al., New Engl. J. Med. 336:1575-1595 (1997)). Whole cell currents are conveniently determined using the standard methodology (see, e.g., Hamil et al., PFlugers. Archiv. 391:85 (1981). Other known assays include: radiolabeled ion flux assays and fluorescence assays using voltage-sensitive dyes (see, e.g., Vestergarrd-Bogind et al., J. Membrane Biol. 88:67-75 (1988); Gonzales & Tsien, Chem. Biol. 4:269-277 (1997); Daniel et al., J. Pharmacol. Meth. 25:185-193 (1991); Holevinsky et al., J. Membrane Biology 137:59-70 (1994)). Generally, the compounds to be tested are present in the range from 1 pM to 100 mM.

The effects of the test compounds upon the function of the polypeptides can be measured by examining any of the parameters described above. Any suitable physiological change that affects GPCR activity can be used to assess the influence of a test compound on the polypeptides of this invention. When the functional consequences are determined using intact cells or animals, one can also measure a variety of effects such as transmitter release, hormone release, transcriptional changes to both known and uncharacterized genetic markers (e.g., northern blots), changes in cell metabolism such as cell growth or pH changes, and changes in intracellular second messengers such as Ca²⁺, IP3 or cAMP.

Preferred assays for G-protein coupled receptors include cells that are loaded with ion or voltage sensitive dyes to report receptor activity. Assays for determining activity of such receptors can also use known agonists and antagonists for other G-protein coupled receptors as negative or positive controls to assess activity of tested compounds. In assays for identifying modulatory compounds (e.g., agonists, antagonists), changes in the level of ions in the cytoplasm or membrane voltage will be monitored using an ion sensitive or membrane voltage fluorescent indicator, respectively. Among the ion-sensitive indicators and voltage probes that may be employed are those disclosed in the Molecular Probes 1997 Catalog. For G-protein coupled receptors, promiscuous G-proteins such as Gα15 and Gα16 can be used in the assay of choice (Wilkie et al., Proc. Nat'l Acad. Sci. USA 88:10049-10053 (1991)). Such promiscuous G-proteins allow coupling of a wide range of receptors.

Receptor activation typically initiates subsequent intracellular events, e.g., increases in second messengers such as IP3, which releases intracellular stores of calcium ions. Activation of some G-protein coupled receptors stimulates the formation of inositol triphosphate (IP3) through phospholipase C-mediated hydrolysis of phosphatidylinositol (Berridge & Irvine, Nature 312:315-21 (1984)). IP3 in turn stimulates the release of intracellular calcium ion stores. Thus, a change in cytoplasmic calcium ion levels, or a change in second messenger levels such as IP3 can be used to assess G-protein coupled receptor function. Cells expressing such G-protein coupled receptors may exhibit increased cytoplasmic calcium levels as a result of contribution from both intracellular stores and via activation of ion channels, in which case it may be desirable although not necessary to conduct such assays in calcium-free buffer, optionally supplemented with a chelating agent such as EGTA, to distinguish fluorescence response resulting from calcium release from internal stores.

Other assays can involve determining the activity of receptors which, when activated, result in a change in the level of intracellular cyclic nucleotides, e.g., cAMP or cGMP, by activating or inhibiting enzymes such as adenylate cyclase. There are cyclic nucleotide-gated ion channels, e.g., rod photoreceptor cell channels and olfactory neuron channels that are permeable to cations upon activation by binding of cAMP or cGMP (see, e.g., Altenhofen et al., Proc. Natl. Acad. Sci. U.S.A. 88:9868-9872 (1991) and Dhallan et al., Nature 347:184-187 (1990)). In cases where activation of the receptor results in a decrease in cyclic nucleotide levels, it may be preferable to expose the cells to agents that increase intracellular cyclic nucleotide levels, e.g., forskolin, prior to adding a receptor-activating compound to the cells in the assay. Cells for this type of assay can be made by co-transfection of a host cell with DNA encoding a cyclic nucleotide-crated ion channel, GPCR phosphatase and DNA encoding a receptor (e.g., certain glutamate receptors, muscarinic acetylcholine receptors, dopamine receptors, serotonin receptors, and the like), which, when activated, causes a change in cyclic nucleotide levels in the cytoplasm.

In a preferred embodiment, IC-GPCR activity is measured by expressing IC-GPCR in a heterologous cell with a promiscuous G-protein that links the receptor to a phospholipase C signal transduction pathway (see Offermanns & Simon, J. Biol. Chem. 270:15175-15180 (1995); see also Example II). Optionally the cell line is HEK-293 (which does not naturally express IC-GPCR) and the promiscuous G-protein is Gα15 (Offermanns & Simon, supra). Modulation of signal transduction is assayed by measuring changes in intracellular Ca²⁺ levels, which change in response to modulation of the IC-GPCR signal transduction pathway via administration of a molecule that associates with IC-GPCR. Changes in Ca²⁺ levels are optionally measured using fluorescent Ca²⁺ indicator dyes and fluorometric imaging.

In one embodiment, the changes in intracellular cAMP or cGMP can be measured using immunoassays. The method described in Offermanns & Simon, J. Biol. Chem. 270:15175-15180 (1995) may be used to determine the level of cAMP. Also, the method described in Felley-Bosco et al., Am. J. Resp. Cell and Mol. Biol. 11:159-164 (1994) may be used to determine the level of cGMP. Further, an assay kit for measuring cAMP and/or cGMP is described in U.S. Pat. No. 4,115,538, herein incorporated by reference.

In another embodiment, phosphatidyl inositol (PI) hydrolysis can be analyzed according to U.S. Pat. No. 5,436,128, herein incorporated by reference. Briefly, the assay involves labeling of cells with ³H-myoinositol for 48 or more hrs. The labeled cells are treated with a test compound for one hour. The treated cells are lysed and extracted in chloroform-methanol-water after which the inositol phosphates were separated by ion exchange chromatography and quantified by scintillation counting. Fold stimulation is determined by calculating the ratio of cpm in the presence of agonist to cpm in the presence of buffer control. Likewise, fold inhibition is determined by calculating the ratio of cpm in the presence of antagonist to cpm in the presence of buffer control (which may or may not contain an agonist).

In another embodiment, transcription levels can be measured to assess the effects of a test compound on signal transduction. A host cell containing the protein of interest is contacted with a test compound for a sufficient time to effect any interactions, and then the level of gene expression is measured. The amount of time to effect such interactions may be empirically determined, such as by running a time course and measuring the level of transcription as a function of time. The amount of transcription may be measured by using any method known to those of skill in the art to be suitable. For example, mRNA expression of the protein of interest may be detected using northern blots or their polypeptide products may be identified using immunoassays. Alternatively, transcription based assays using reporter gene may be used as described in U.S. Pat. No. 5,436,128, herein incorporated by reference. The reporter genes can be, e.g., chloramphenicol acetyltransferase, firefly luciferase, bacterial luciferase, β-galactosidase and alkaline phosphatase. Furthermore, the protein of interest can be used as an indirect reporter via attachment to a second reporter such as green fluorescent protein (see, e.g., Mistili & Spector, Nature Biotechnology 15:961-964 (1997)).

The amount of transcription is then compared to the amount of transcription in either the same cell in the absence of the test compound, or it may be compared with the amount of transcription in a substantially identical cell that lacks the protein of interest. A substantially identical cell may be derived from the same cells from which the recombinant cell was prepared but which had not been modified by introduction of heterologous DNA. Any difference in the amount of transcription indicates that the test compound has in some manner altered the activity of the protein of interest.

B. Modulators

The compounds tested as modulators of IC-GPCR can be any small chemical compound, or a biological entity, such as a protein, sugar, nucleic acid or lipid. Alternatively, modulators can be genetically altered versions of IC-GPCR. Typically, test compounds will be small chemical molecules and peptides. Essentially any chemical compound can be used as a potential modulator or ligand in the assays of the invention, although most often compounds can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one preferred embodiment, high throughput screening methods involve providing a combinatorial chemical or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, Jan. 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

C. Solid State and Soluble High Throughput Assays

In one embodiment the invention provide soluble assays using molecules such as a domain such as ligand binding domain, an extracellular domain, a transmembrane domain (e.g., one comprising seven transmembrane regions and cytosolic loops), the transmembrane domain and a cytoplasmic domain, an active site, a subunit association region, etc.; a domain that is covalently linked to a heterologous protein to create a chimeric molecule; IC-GPCR; or a cell or tissue expressing IC-GPCR, either naturally occurring or recombinant. In another embodiment, the invention provides solid phase based in vitro assays in a high throughput format, where the domain, chimeric molecule, IC-GPCR, or cell or tissue expressing IC-GPCR is attached to a solid phase substrate.

In the high throughput assays of the invention, it is possible to screen up to several thousand different modulators or ligands in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100- about 1500 different compounds. It is possible to assay several different plates per day; assay screens for up to about 6,000-20,000 different compounds is possible using the integrated systems of the invention. More recently, microfluidic approaches to reagent manipulation have been developed, e.g., by Caliper Technologies (Palo Alto, Calif.).

The molecule of interest can be bound to the solid state component, directly or indirectly, via covalent or non covalent linkage e.g., via a tag. The tag can be any of a variety of components. In general, a molecule which binds the tag (a tag binder) is fixed to a solid support, and the tagged molecule of interest (e.g., the signal transduction molecule of interest) is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, etc.) Antibodies to molecules with natural binders such as biotin are also widely available and appropriate tag binders; see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody which recognizes the first antibody. In addition to antibody-antigen interactions, receptor-ligand interactions are also appropriate as tag and tag-binder pairs. For example, agonists and antagonists of cell membrane receptors (e.g., cell receptor-ligand interactions such as transferrin, c-kit, viral receptor ligands, cytokine receptors, chemokine receptors, interleukin receptors, immunoglobulin receptors and antibodies, the cadherein family, the integrin family, the selectin family, and the like; see, e.g., Pigott & Power, The Adhesion Molecule Facts Book I (1993). Similarly, toxins and venoms, viral epitopes, hormones (e.g., opiates, steroids, etc.), intracellular receptors (e.g. which mediate the effects of various small ligands, including steroids, thyroid hormone, retinoids and vitamin D; peptides), drugs, lectins, sugars, nucleic acids (both linear and cyclic polymer configurations), oligosaccharides, proteins, phospholipids and antibodies can all interact with various cell receptors.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly gly sequences of between about 5 and 200 amino acids. Such flexible linkers are known to persons of skill in the art. For example, poly(ethelyne glycol) linkers are available from Shearwater Polymers, Inc. Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature. See, e.g., Merrifield, J. Am. Chem. Soc. 85:2149-2154 (1963) (describing solid phase synthesis of, e.g., peptides); Geysen et al., J. Immun. Meth. 102:259-274 (1987) (describing synthesis of solid phase components on pins); Frank & Doring, Tetrahedron 44:60316040 (1988) (describing synthesis of various peptide sequences on cellulose disks); Fodor et al., Science, 251:767-777 (1991); Sheldon et al., Clinical Chemistry 39(4):718-719 (1993); and Kozal et al., Nature Medicine 2(7):753759 (1996) (all describing arrays of biopolymers fixed to solid substrates). Non-chemical approaches for fixing tag binders to substrates include other common methods, such as heat, cross-linking by UV radiation, and the like.

D. Computer-Based Assays

Yet another assay for compounds that modulate IC-GPCR activity involves computer assisted drug design, in which a computer system is used to generate a three-dimensional structure of IC-GPCR based on the structural information encoded by the amino acid sequence. The input amino acid sequence interacts directly and actively with a preestablished algorithm in a computer program to yield secondary, tertiary, and quaternary structural models of the protein. The models of the protein structure are then examined to identify regions of the structure that have the ability to bind, e.g., ligands. These regions are then used to identify ligands that bind to the protein.

The three-dimensional structural model of the protein is generated by entering protein amino acid sequences of at least 10 amino acid residues or corresponding nucleic acid sequences encoding a IC-GPCR polypeptide into the computer system. The amino acid sequence of the polypeptide of the nucleic acid encoding the polypeptide is selected from the group consisting of SEQ ID NOS:1 or 2 or SEQ ID NOS:3 or 4, and conservatively modified versions thereof. The amino acid sequence represents the primary sequence or subsequence of the protein, which encodes the structural information of the protein. At least 10 residues of the amino acid sequence (or a nucleotide sequence encoding 10 amino acids) are entered into the computer system from computer keyboards, computer readable substrates that include, but are not limited to, electronic storage media (e.g., magnetic diskettes, tapes, cartridges, and chips), optical media (e.g., CD ROM), information distributed by internet sites, and by RAM. The three-dimensional structural model of the protein is then generated by the interaction of the amino acid sequence and the computer system, using software known to those of skill in the art.

The amino acid sequence represents a primary structure that encodes the information necessary to form the secondary, tertiary and quaternary structure of the protein of interest. The software looks at certain parameters encoded by the primary sequence to generate the structural model. These parameters are referred to as “energy terms,” and primarily include electrostatic potentials, hydrophobic potentials, solvent accessible surfaces, and hydrogen bonding. Secondary energy terms include van der Waals potentials. Biological molecules form the structures that minimize the energy terms in a cumulative fashion. The computer program is therefore using these terms encoded by the primary structure or amino acid sequence to create the secondary structural model.

The tertiary structure of the protein encoded by the secondary structure is then formed on the basis of the energy terms of the secondary structure. The user at this point can enter additional variables such as whether the protein is membrane bound or soluble, its location in the body, and its cellular location, e.g., cytoplasmic, surface, or nuclear. These variables along with the energy terms of the secondary structure are used to form the model of the tertiary structure. In modeling the tertiary structure, the computer program matches hydrophobic faces of secondary structure with like, and hydrophilic faces of secondary structure with like.

Once the structure has been generated, potential ligand binding regions are identified by the computer system. Three-dimensional structures for potential ligands are generated by entering amino acid or nucleotide sequences or chemical formulas of compounds, as described above. The three-dimensional structure of the potential ligand is then compared to that of the IC-GPCR protein to identify ligands that bind to IC-GPCR. Binding affinity between the protein and ligands is determined using energy terms to detennine which ligands have an enhanced probability of binding to the protein.

Computer systems are also used to screen for mutations, polymorphic variants, alleles and interspecies homologs of IC-GPCR genes. Such mutations can be associated with disease states or genetic traits. As described above, GENECHIP® and related technology can also be used to screen for mutations, polymorphic variants, alleles and interspecies homologs. Once the variants are identified, diagnostic assays can be used to identify patients having such mutated genes. Identification of the mutated IC-GPCR genes involves receiving input of a first amino acid or nucleic acid sequence encoding IC-GPCR, selected from the group consisting of SEQ ID NOS:1 and 2, or SEQ ID NOS:3 and 4 and conservatively modified versions thereof. The sequence is entered into the computer system as described above. The first nucleic acid or amino acid sequence is then compared to a second nucleic acid or amino acid sequence that has substantial identity to the first sequence. The second sequence is entered into the computer system in the manner described above. Once the first and second sequences are compared, nucleotide or amino acid differences between the sequences are identified. Such sequences can represent allelic differences in IC-GPCR genes, and mutations associated with disease states and genetic traits.

### VI. Kits

IC-GPCR and its homologs are a useful tool for identifying islet cells, for forensics and paternity determinations, and for examining islet cell signal transduction. IC-GPCR specific reagents that specifically hybridize to IC-GPCR nucleic acid, such as IC-GPCR probes and primers, and IC-GPCR specific reagents that specifically bind to the IC-GPCR protein, e.g., IC-GPCR antibodies are used to examine islet cell expression, signal transduction regulation and diagnose diabetes.

Nucleic acid assays for the presence of IC-GPCR DNA and RNA in a sample include numerous techniques are known to those skilled in the art, such as Southern analysis, northern analysis, dot blots, RNase protection, S1 analysis, amplification techniques such as PCR and LCR, and in situ hybridization. In in situ hybridization, for example, the target nucleic acid is liberated from its cellular surroundings in such as to be available for hybridization within the cell while preserving the cellular morphology for subsequent interpretation and analysis (see Example I). The following articles provide an overview of the art of in situ hybridization: Singer et al., Biotechniques 4:230-250 (1986); Haase et al., Methods in Virology, vol. VII, pp. 189-226 (1984); and Nucleic Acid Hybridization: A Practical Approach (Hames et al., eds. 1987). In addition, IC-GPCR protein can be detected with the various immunoassay techniques described above. The test sample is typically compared to both a positive control (e.g., a sample expressing recombinant IC-GPCR) and a negative control.

The present invention also provides for kits for screening for modulators of IC-GPCR. Such kits can be prepared from readily available materials and reagents. For example, such kits can comprise any one or more of the following materials: IC-GPCR, reaction tubes, and instructions for testing IC-GPCR activity. Optionally, the kit contains biologically active IC-GPCR. A wide variety of kits and components can be prepared according to the present invention, depending upon the intended user of the kit and the particular needs of the user.

### VII. Administration and Pharmaceutical Compositions

IC-GPCR signal modulators can be administered directly to the mammalian subject for modulation of islet cell signaling in vivo. Administration is by any of the routes normally used for introducing a modulator compound into ultimate contact with the tissue to be treated, optionally the tongue or mouth. The signal modulators are administered in any suitable manner, optionally with pharmaceutically acceptable carriers. Suitable methods of administering such modulators are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions of the present invention (see, e.g., Remington's Pharmaceutical Sciences, 17th ed. (1985)).

The signal modulators, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for administration include aqueous and non-aqueous solutions, isotonic sterile solutions, which can contain antioxidants, buffers, bacteriostats, and solutes that render the formulation isotonic, and aqueous and non-aqueous sterile suspensions that can include suspending agents, solubilizers, thickening agents, stabilizers, and preservatives. In the practice of this invention, compositions can be administered, for example, by orally, topically, intravenously, intraperitoneally, intravesically or intrathecally. Optionally, the compositions are administered orally or nasally. The formulations of compounds can be presented in unit-dose or multi-dose sealed containers, such as ampules and vials. Solutions and suspensions can be prepared from sterile powders, granules, and tablets of the kind previously described. The modulators can also be administered as part a of prepared food or drug.

The dose administered to a patient, in the context of the present invention should be sufficient to effect a beneficial response in the subject over time. The dose will be determined by the efficacy of the particular signal modulators employed and the condition of the subject, as well as the body weight or surface area of the area to be treated. The size of the dose also will be determined by the existence, nature, and extent of any adverse side-effects that accompany the administration of a particular compound or vector in a particular subject.

In determining the effective amount of the modulator to be administered in a physician may evaluate circulating plasma levels of the modulator, modulator toxicities, and the production of anti-modulator antibodies. In general, the dose equivalent of a modulator is from about 1 ng/kg to 10 mg/kg for a typical subject.

For administration, signal modulators of the present invention can be administered at a rate determined by the LD-50 of the modulator, and the side-effects of the inhibitor at various concentrations, as applied to the mass and overall health of the subject. Administration can be accomplished via single or divided doses.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

### EXAMPLES

The following examples are offered to illustrate, but not to limit the claimed invention.

### Example I

Cloning and Expression of IC-GPCR

This example describes the discovery of new members of the GPCR superfamily of proteins that is highly abundant in pancreatic islets and whose signal transduction modulation is important for the treatment of diabetes. cDNA libraries prepared from RNA obtained from isolated murine islets and from murine islet cell lines were used to isolate the murine IC-GPCR nucleic acids of the invention. RNA isolated from human islets were used to isolate the human IC-GPCR nucleic acids of the invention.

Islets were isolated from murine pancreas, RNA was extracted and used to prepare double stranded cDNA using standard techniques (see e.g. Sambrook et al., supra and Ausubel et al., supra). The cDNA was cloned into the pZL1 vector (Invitrogen) and the 3′ ends of individual clones were sequenced in multiple rounds of sequencing reactions. Sequence data representing 12,000 independent clones were used to construct oligonucleotide probes synthesized on a GENECHIP® (Affymetrix Inc., Santa Clara, Calif.), producing a murine islet chip. Islet cell cDNA clones were then hybridized to the oligonucleotide arrays and specific clones were identified.

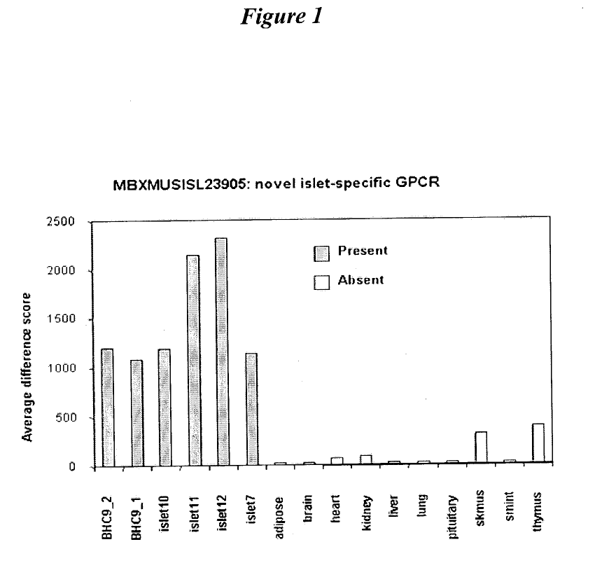

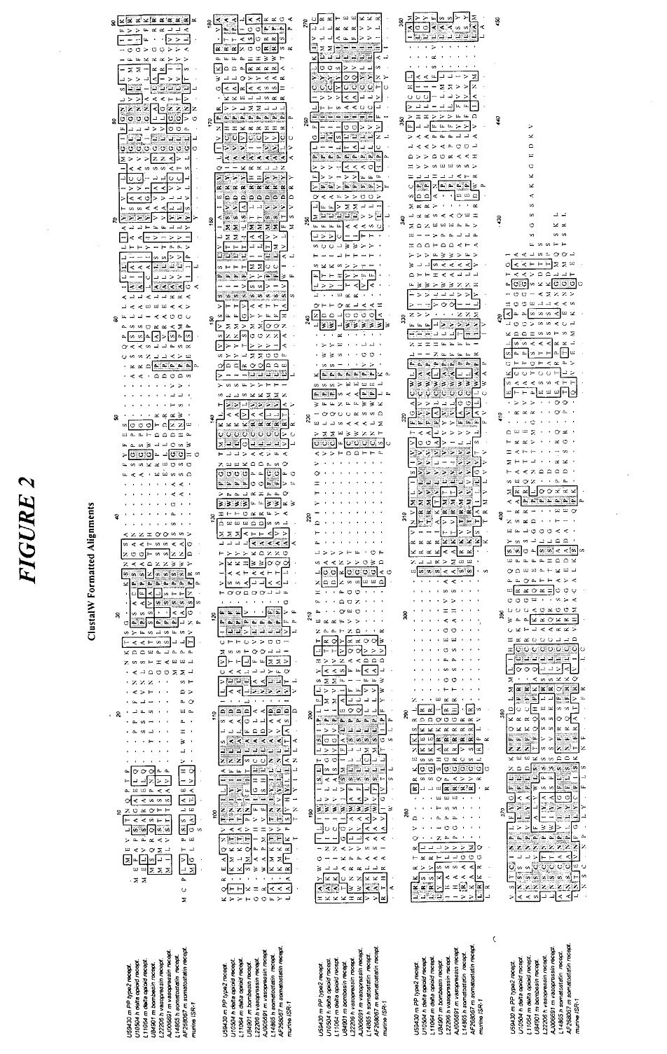

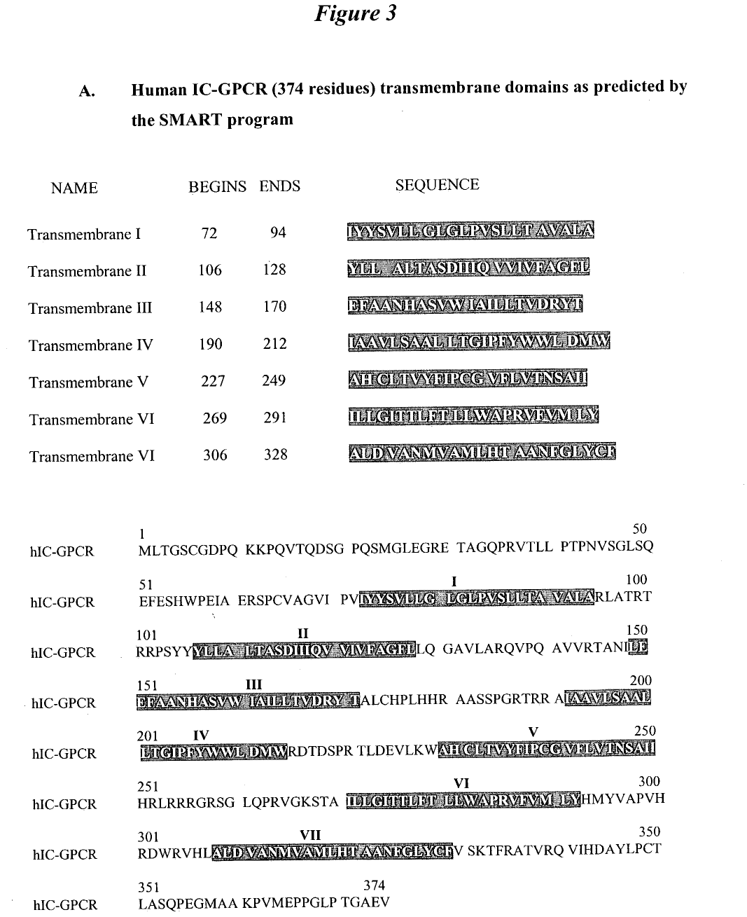

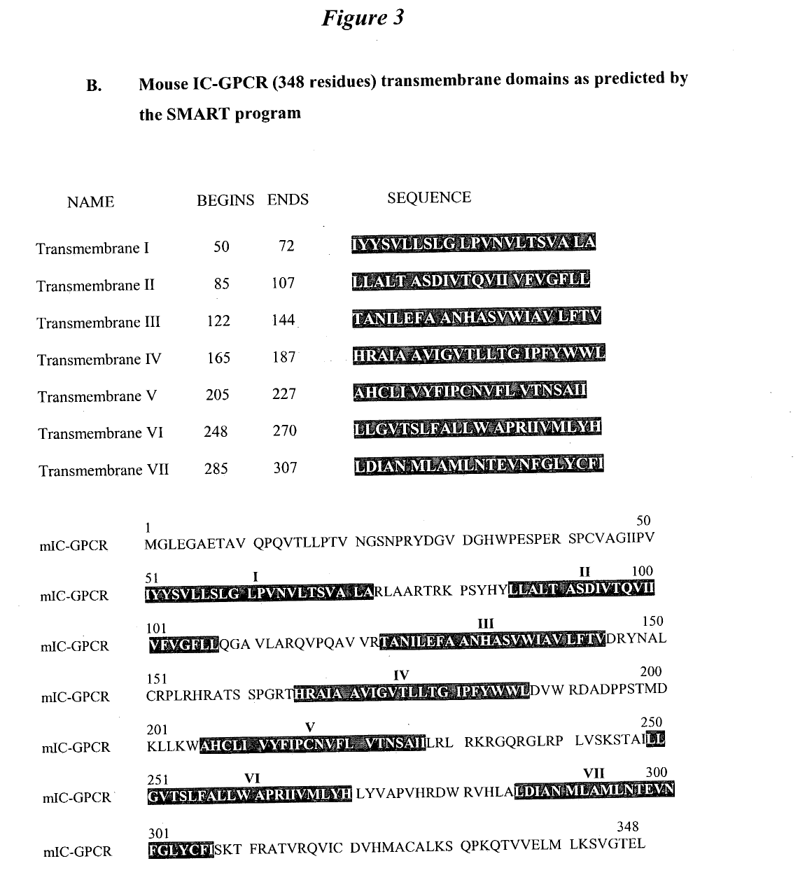

Probe set MBXMUSISL23905 was hybridized to the murine islet cell chip and found to be highly islet cell specific. The MBXMUSISL23905 probe set was hybridized to RNA isolated from the murine pancreatic beta cell line BHC9, RNA isolated from four different islet cell preparations and RNA isolated from ten other tissues. The results shown in FIG. 1 revealed that the MBXMUSISL23905 probe set is highly expressed in BHC9 cells and pancreatic islets with an average difference score ranging from ≧1000 to 2500 whereas the average difference score in the other ten tissues ranged from 0 to ≦500. This represents an extremely islet specific expression pattern. cDNA clones corresponding to this probe set were sequenced from the islet cDNA library. The murine IC-GPCR clone provided in SEQ ID NO:4 was identified. The murine IC-GPCR clone contains an open reading frame of 1067 nucleotides encoding a protein of 348 amino acids. Analysis of the predicted amino acid sequence of the murine IC-GPCR clone provided in SEQ ID NO:2 indicates that the clone contains features typical of G-protein coupled receptors such as a seven transmembrane domains and homology with several members of the GPCR superfamily, including the vasopressin, somatostatin, delta opioid, bombesin and pancreatic polypeptide receptors. See FIG. 2 illustrating an alignment of the murine IC-GPCR amino acid sequence to members of the GPCR superfamily of proteins and FIG. 3 showing the predicted 7 transmembrane domains of the human (see below) and murine IC-GPCR (as predicted by the SMART software: Simple Modular Architecture Research Tool, publicly available at: http:/smart.embl-heidelberg.de).

The murine IC-GPCR amino acid sequence was used to screen in-house and public databases including Genbank, Swissprot, dbEST, the HTG, the homo sapiens genomic contig sequences and the golden path UC Santa Cruz. This BLAST search identified a single sequence in the human genomic contig sequences, HTG and the golden path human genomic databases. The corresponding genomic clone ID is Hs17_{—}10829. A comparison of the human and murine IC-GPCR amino acid sequences revealed that the human clone present in the publicly available data bases is not a full length sequence.

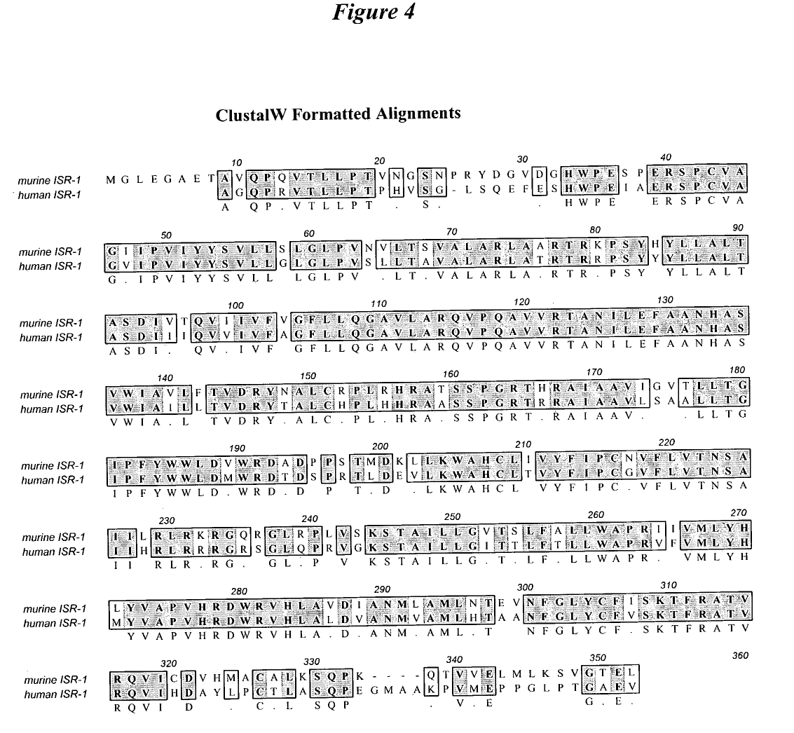

In order to obtain nucleotide sequences-encoding the full length human IC-GPCR protein, PCR primers were designed to recognize the published human IC-GPCR sequence. RT-PCR was performed using human islet RNA and standard techniques (see e.g. Sambrook et al., supra and Ausubel et al., supra). The nucleotide sequence of the resulting full length human IC-GPCR clone is provided in SEQ ID NO:3 and the amino acid sequence of the encoded protein in provided in SEQ ID NO:1. A ClustalW alignment of the murine and human IC-GPCR amino acid sequences shown in FIG. 4 indicates that the proteins are highly similar sharing 72% identical and 84% similar residues.

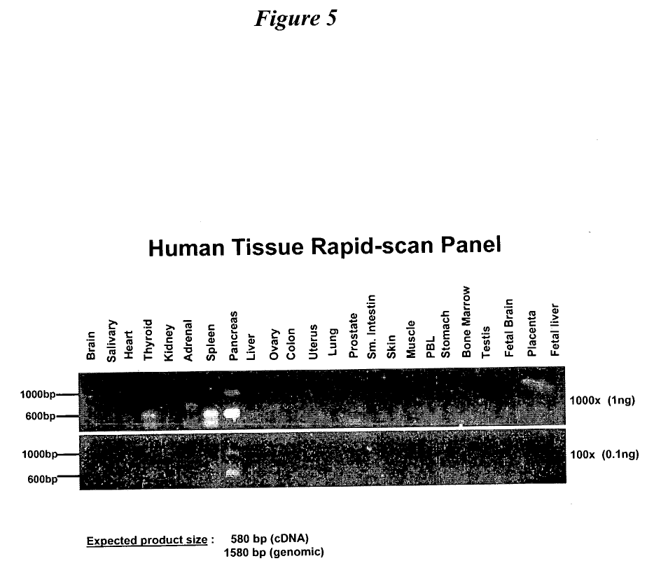

The tissue expression pattern of the human IC-GPCR gene was examined by RT-PCR using a human tissue rapid scan panel of RNA obtained (Origene) and primers corresponding to nucleotides 5′GCTGAGCCAGGAGTTTGAAAGC3′ (SEQ ID NO:5) (sense) and 5′CACGCCACAAGGGATGAAATAG3′(SEQ ID NO:6) (antisense). The results are shown in FIG. 5 and are similar to the expression pattern observed in murine tissue discussed above. Either 1 ηg or 0.1 ηg of cDNA was used for the PCR reaction. High levels of human IC-GPCR amplified product were observed in pancreas and lower levels of amplified product were observed in spleen and thymus when the higher amount of cDNA was added to the PCR reaction. Only pancreatic expression was observed when the lower amount of cDNA was added to the PCR reaction.

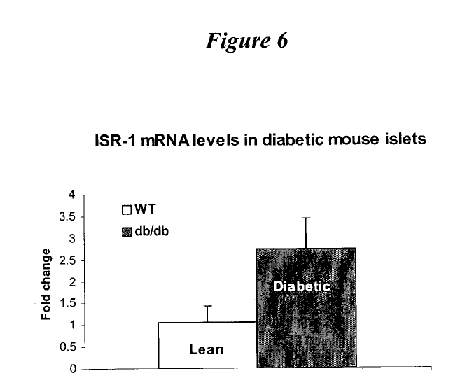

The expression of murine IC-GPCR in diabetic mice was determined. The murine db mutation is a well characterized animal model for type II diabetes. The db/db animals used in the experiment were frankly diabetic with mean blood glucose levels of >450 mg/dl (heterozygous age matched controls had blood glucose levels of <150 mg/dl). Mouse islets were isolated from 12 week old heterozygous (db/+) or diabetic (db/db) animals. Islets were resuspended in TRIZOL® solution (Brinkman Instruments, Inc.) and RNA was extracted according to manufacturer's instructions. For each sample, 2 μg of total RNA was reverse transcribed to generate cDNA using standard protocols (see e.g. Sambrook et al., supra and Ausubel et al., supra). Murine IC-GPCR mRNA levels were measured by real-time PCR using murine specific primers corresponding to nucleotides 5′CGGGTCTTCGTCATGCTCTA3′ (SEQ ID NO:7) (sense) and 5′GGAAAGTCTTGCTGACAAAGCAGTA3′ (SEQ ID NO:8) (antisense) and SYBR® green I dye (Applied Biosystems). Direct detection of PCR product was monitored by measuring the increase in fluorescence caused by the binding of SYBR® green dye to double stranded DNA used according to manufacturer's instructions. The fluorescent signal in each sample was normalized to a corresponding β-actin signal (endogenous control). As shown in FIG. 6, IC-GPCR levels are significantly elevated in diabetic islets (mean fold change±SD, n=3, p<0.05) relative to the heterozygous (db/+) controls.

### Example II

Generation and Use of Anti-Human and Anti-Mouse IC-GPCR Antibodies

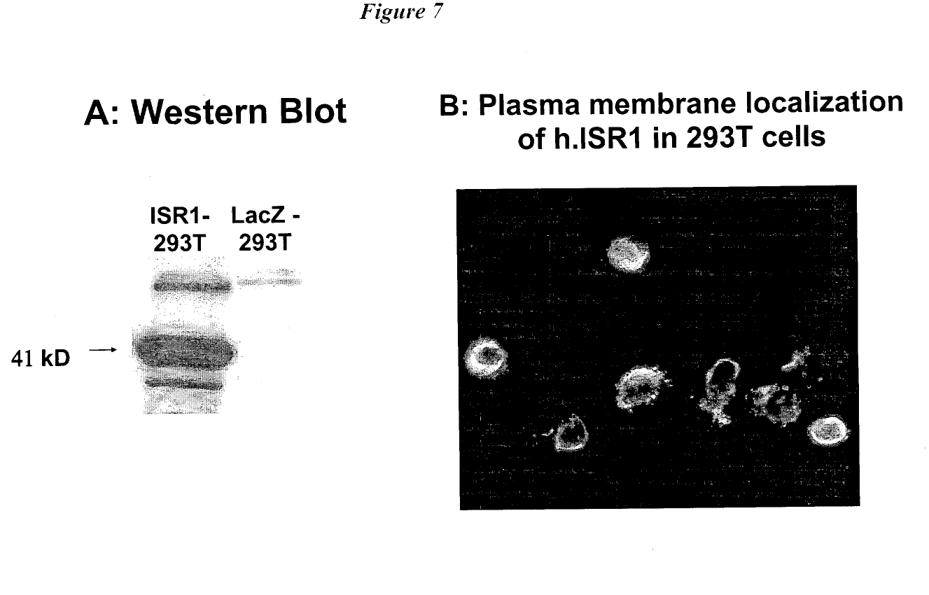

An antibody to human IC-GPCR detects the recombinant protein by Western blot and by immunocytochemistry. Antibody h.ISR1 was raised to the C-terminal peptide of human IC-GPCR, spanning amino acids CPVMEPPGLPTGAEV of SEQ ID NO:1, and affinity purified on the same peptide. Western blots using this antibody detect a prominent band at the expected molecular weight of 41 kilodaltons in HEK293T fibroblasts transfected with a mammalian expression vector containing the human IC-GPCR cDNA (SEQ ID NO:3), but no such band is detected in cells transfected with a control vector expressing Lac Z (FIG. 7). Immunocytochemical detection of human IC-GPCR transfected HEK293T cells shows that most of the human IC-GPCR is localized at the plasma membrane. Thus, this antibody is able to specifically detect human IC-GPCR in cell lysates after denaturation and in also whole cells.

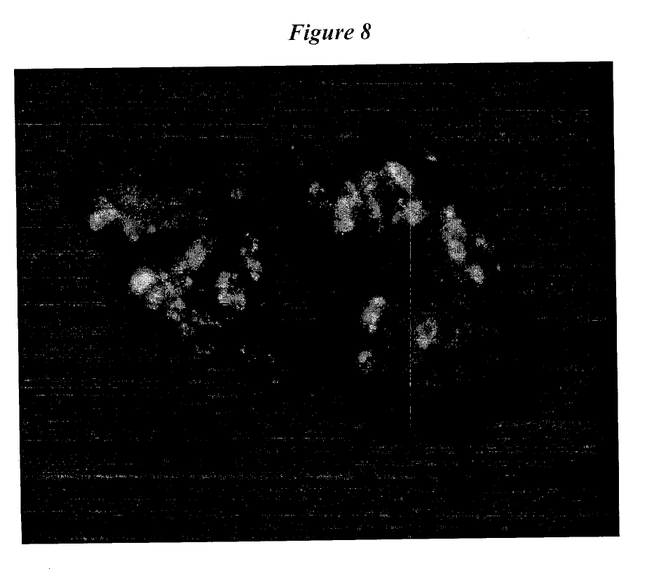

The antibody described above was used to demonstrate that IC-GPCR protein is abundant in human islet cells. Immunofluorescent staining demonstrates native IC-GPCR protein expression in most islet cells in each islet of a human pancrease tissue section (FIG. 8). Some cells appear to have a more prominent fluorescence than others within the islet; this may be due to greater abundance in some cells or it may be due to the distribution of the protein within the cell and the disposition of that cell within the section.

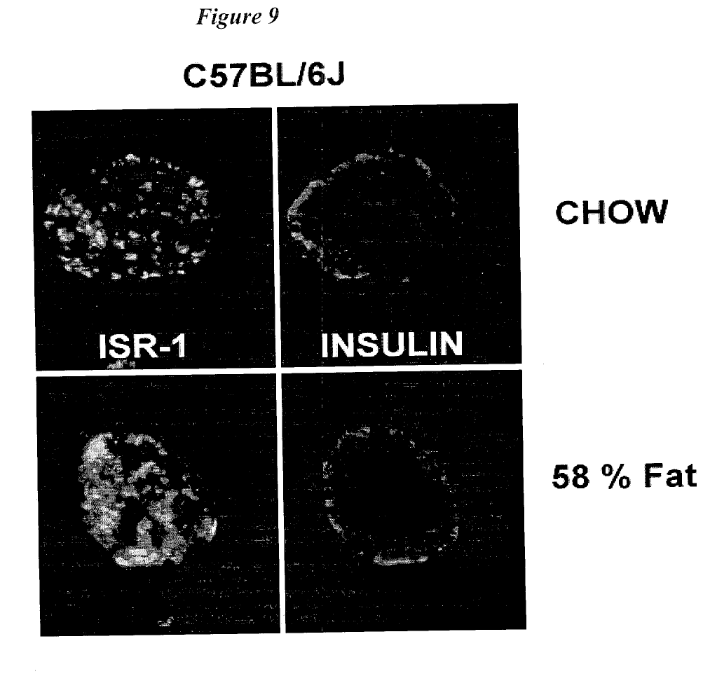

An antibody to mouse IC-GPCR detects the native protein in mouse pancreatic tissue slices. Antibody m.ISR-1 was raised against the C-terminal peptide of mouse IC-GPCR, spanning amino acids CTVVELMLKSVGTEL of SEQ ID NO:2, and was used to localize the mouse IC-GPCR protein to beta cells within mouse pancreatic tissue section. The anti-mouse IC-GPCR antibody shows that the IC-GPCR protein is found in the vast majority of insulin positive cells (FIG. 9). No IC-GPCR is detected in surrounding acinar tissue. The fluorescence intensity of anti-IC-GPCR staining is greatly increased in islets from mice fed a 58% diet for 14 weeks relative to islets from mice fed a chow diet. This finding probably reflects an induction of IC-GPCR protein expression in high fat fed mice.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. An isolated nucleic acid encoding a G-protein coupled receptor, the receptor comprising greater than 70% amino acid identity to an amino acid sequence of SEQ ID NO:1, orSEQ ID NO:2.

2. The isolated nucleic acid of claim 1, wherein the nucleic acid encodes a receptor that specifically binds to polyclonal antibodies generated against SEQ ID NO:1, or SEQ ID NO:2.

3. The isolated nucleic acid of claim 1, wherein the nucleic acid encodes a receptor that has G-protein coupled receptor activity.

4. The isolated nucleic acid of claim 1, wherein the nucleic acid encodes a receptor comprising an amino acid sequence of SEQ ID NO:1, or SEQ ID NO:2.

5. The isolated nucleic acid sequence of claim 1, wherein the nucleic acid comprises a nucleotide sequence of SEQ ID NO:3, or SEQ ID NO:4.

6. The isolated nucleic acid of claim 1, wherein the nucleic acid is from a human, or a mouse.

7. The isolated nucleic acid of claim 1, wherein the nucleic acid encodes a receptor having a molecular weight of about between 38-42 kDa.

8. An isolated nucleic acid encoding a G-protein coupled receptor, wherein the nucleic acid specifically hybridizes under highly stringent conditions to a nucleic acid having the sequence of SEQ ID NO:3, or SEQ ID NO:4.

9. An isolated nucleic acid encoding a G-protein coupled receptor, the receptor comprising greater than 70% amino acid identity to a polypeptide having a sequence of SEQ ID NO:1, or SEQ ID NO:2, wherein the nucleic acid selectively hybridizes under moderately stringent hybridization conditions to a nucleotide sequence of SEQ ID NO:3, or SEQ ID NO:4.

10. An isolated nucleic acid encoding an extracellular domain of a G-protein coupled receptor, the extracellular domain having greater than 70% amino acid sequence identity to the extracellular domain of SEQ ID NO:1.

11. The isolated nucleic acid of claim 10, wherein the nucleic acid encodes the extracellular domain linked to a nucleic acid encoding a heterologous polypeptide, forming a chimeric polypeptide.

12. The isolated nucleic acid of claim 10, wherein the nucleic acid encodes the extracellular domain of SEQ ID NO:1.

13. An isolated nucleic acid encoding a transmembrane domain of a G-protein coupled receptor, the transmembrane domain comprising greater than 70% amino acid sequence identity to the transmembrane domain of SEQ ID NO:1.

14. The isolated nucleic acid of claim 13, wherein nucleic acid encodes the transmembrane domain linked to a nucleic acid encoding a heterologous polypeptide, forming a chimeric polypeptide.

15. The isolated nucleic acid of claim 13, wherein the nucleic acid encodes the transmembrane domain of SEQ ID NO:1.

16. The isolated nucleic acid of claim 13, wherein the nucleic acid further encodes a cytoplasmic domain comprising greater than 70% amino acid identity to the cytoplasmic domain of SEQ ID NO:1.

17. The isolated nucleic acid of claim 16, wherein the nucleic acid encodes the cytoplasmic domain of SEQ ID NO:1.

18. An isolated G-protein coupled receptor, the receptor comprising greater than 70% amino acid sequence identity to an amino acid sequence of SEQ ID NO:1, or SEQ ID NO:2.

19. The isolated receptor of claim 18, wherein the receptor specifically binds to polyclonal antibodies generated against SEQ ID NO:1, or SEQ ID NO:2.

20. The isolated receptor of claim 18, wherein the receptor has G-protein coupled receptor activity.

21. The isolated receptor of claim 18, wherein the receptor has an amino acid sequence of SEQ ID NO:1, or SEQ ID NO:2.

22. The isolated receptor of claim 18, wherein the receptor is from a human, or a murine.

23. An isolated polypeptide comprising an extracellular domain of a G-protein coupled receptor, the extracellular domain comprising greater than 70% amino acid sequence identity to the extracellular domain of SEQ ID NO:1.

24. The isolated polypeptide of claim 23, wherein the polypeptide encodes the extracellular domain of SEQ ID NO:1.

25. The isolated polypeptide of claim 23, wherein the extracellular domain is covalently linked to a heterologous polypeptide, forming a chimeric polypeptide.

26. An isolated polypeptide comprising a transmembrane domain of a G-protein coupled receptor, the transmembrane domain comprising greater than 70% amino acid sequence identity to the transmembrane domain of SEQ ID NO:1.

27. The isolated polypeptide of claim 26, wherein the polypeptide encodes the transmembrane domain of SEQ ID NO:1.

28. The isolated polypeptide of claim 26, further comprising a cytoplasmic domain comprising greater than 70% amino acid identity to the cytoplasmic domain of SEQ ID NO:1.

29. The isolated polypeptide of claim 28, wherein the polypeptide encodes the cytoplasmic domain of SEQ ID NO:1.

30. The isolated polypeptide of claim 26, wherein the transmembrane domain is covalently linked to a heterologous polypeptide, forming a chimeric polypeptide.

31. The isolated polypeptide of claim 30, wherein the chimeric polypeptide has G-protein coupled receptor activity.

32. An antibody that selectively binds to the receptor of claim 18.

33. An expression vector comprising the nucleic acid of claim 1.

34. A host cell transfected with the vector of claim 33.

35. A method for identifying a compound that modulates signaling in islet cells, the method comprising the steps of:
(i) contacting the compound with a polypeptide comprising an extracellular domain of a G-protein coupled receptor, the extracellular domain comprising greater than 70% amino acid sequence identity to the extracellular domain of SEQ ID NO:1, or SEQ ID NO:2; and
(ii) determining the functional effect of the compound upon the extracellular domain.

36. The method of claim 35, wherein the polypeptide is a G-protein coupled receptor, the receptor comprising greater than 70% amino acid identity to a polypeptide encoding SEQ ID NO:1, or SEQ ID NO:2.

37. The method of claim 36, wherein polypeptide comprises an extracellular domain that is covalently linked to a heterologous polypeptide, forming a chimeric polypeptide.

38. The method of claim 36 or 37, wherein the polypeptide has G-protein coupled receptor activity.

39. The method of claim 35, wherein the extracellular domain is linked to a solid phase.

40. The method of claim 39, wherein the extracellular domain is covalently linked to a solid phase.

41. The method of claim 36 or 37, wherein the functional effect is determined by measuring changes in intracellular cAMP, IP3, or Ca²⁺.

42. The method of claim 35, wherein the functional effect is a chemical effect.

43. The method of claim 35, wherein the functional effect is determined by measuring binding of the compound to the extracellular domain.

44. The method of claim 35, wherein the polypeptide is recombinant.

45. The method of claim 35, wherein the polypeptide is from a murine, or a human.

46. The method of claim 35, wherein the polypeptide comprises an amino acid sequence of SEQ ID NO:1, or SEQ ID NO:2.

47. The method of claim 36 or 37, wherein the polypeptide is expressed in a cell or cell membrane.

48. The method of claim 47, wherein the cell is a eukaryotic cell.

49. The method of claim 48, wherein the eukaryotic cell is an islet cell.

50. A method for identifying a compound that modulates signaling in islet cells, the method comprising the steps of:
(i) contacting the compound with a polypeptide comprising a transmembrane domain of a G-protein coupled receptor, the transmembrane domain comprising greater than 70% amino acid sequence identity to the transmembrane domain of SEQ ID NO:1, or SEQ ID NO:2; and
(ii) determining the functional effect of the compound upon the transmembrane domain.

51. The method of claim 50, wherein the polypeptide comprises an transmembrane domain that is covalently linked to a heterologous polypeptide, forming a chimeric polypeptide.

52. The method of claim 50, wherein the chimeric polypeptide has G-protein coupled receptor activity.

53. The method of claim 50, wherein the functional effect is determined by measuring changes in intracellular cAMP, IP3, or Ca²⁺.

54. The method of claim 50, wherein the functional effect is a chemical effect.

55. The method of claim 50, wherein the functional effect is a physical effect.

56. The method of claim 50, wherein the polypeptide is recombinant.

57. The method of claim 50, wherein the polypeptide is from a murine, or a human.

58. The method of claim 50 or 51, wherein the polypeptide is expressed in a cell or cell membrane.

59. The method of claim 58, wherein the cell is a eukaryotic cell.

60. The method of claim 59, wherein the eukaryotic cell is an islet cell.

61. A method for treating a patient diagnosed with type I or type II diabetes, the method comprising administering a therapeutically effective amount of the compound identified by the method of claim 35 or claim 50.

62. A method of making a G-protein coupled receptor, the method comprising the step of expressing the receptor from a recombinant expression vector comprising a nucleic acid encoding the receptor, wherein the amino acid sequence of the receptor comprises greater than 70% amino acid identity to a polypeptide having a sequence of SEQ ID NO:1, or SEQ ID NO:2.

63. A method of making a recombinant cell comprising a G-protein coupled receptor, the method comprising the step of transducing the cell with an expression vector comprising a nucleic acid encoding the receptor, wherein the amino acid sequence of the receptor comprises greater than 70% amino acid identity to a polypeptide having a sequence of SEQ ID NO:1, or SEQ ID NO:2.

64. A method of making a recombinant expression vector comprising a nucleic acid encoding a G-protein coupled receptor, the method comprising the step of ligating to an expression vector a nucleic acid encoding the receptor, wherein the amino acid sequence of the receptor comprises greater than 70% amino acid identity to a polypeptide having a sequence of SEQ ID NO:1, or SEQ ID NO:2.

65. A method of diagnosing type I or type II diabetes or a predisposition for type I or type II diabetes in a patient, said method comprising:
detecting the level of a polypeptide at least 70% identical to SEQ ID NOs: 1 or 2 in a sample from a patient, wherein a high level of the polypeptide in the sample, compared to level in a non-diabetic individual indicates that the patient is diabetic or is predisposed to some pathological aspects of diabetes.

66. A method of diagnosing type I or type II diabetes or a predisposition for type I or type II diabetes in a patient, said method comprising:
detecting the level of activity of a polypeptide at least 70% identical to SEQ ID NOs: 1 or 2 in a sample from a patient, wherein a high level of activity of the polypeptide in the sample, compared to the level of activity in a non-diabetic individual indicates that the patient is diabetic or is predisposed to some pathological aspects of diabetes.


SEQUENCE LISTING (59 sequences)

SEQ ID NO: 1 - protein, 374 aa, Homo sapiens [sequence omitted]

SEQ ID NO: 2 - protein, 348 aa, Mus musculus
  murine islet cell G-protein coupled receptor (IC-GPCR)
MGLEGAETAVQPQVTLLPTVNGSNPRYDGVDGHWPESPERSPCVAGIIPVIYYSVLLSLGLPVNVLTSVALARLAARTRKPSYHYLLALTASDIVTQVIIVFVGFLLQGAVLARQVPQAVVRTANILEFAANHASVWIAVLFTVDRYNALCRPLRHRATSSPGRTHRAIAAVIGVTLLTGIPFYWWLDVWRDADPPSTMDKLLKWAHCLIVYFIPCNVFLVTNSAIILRLRKRGQRGLRPLVSKSTAILLGVTSLFALLWAPRIIVMLYHLYVAPVHRDWRVHLALDIANMLAMLNTEVNFGLYCFISKTFRATVRQVICDVHMACALKSQPKQTVVELMLKSVGTEL

SEQ ID NO: 3 - DNA, 1793 bp, Homo sapiens
  human islet cell G-protein coupled receptor (IC-GPCR)
aaacaaccacttggagagccaaggggtgagaggtacagctggcgatgctgacagggagctgcggggaccctcagaaaaagccacaggtgacccaggactcagggccccagagcatggggcttgagggacgagagacagctggccagccacgagtgaccctgctgcccacgcccaacgtcagcgggctgagccaggagtttgaaagccactggccagagatcgcagagaggtccccgtgtgtggctggcgtcatccctgtcatctactacagtgtcctgctgggcttggggctgcctgtcagcctcctgaccgcagtggccctggcgcgccttgccaccaggaccaggaggccctcctactactaccttctggcgctcacagcctcggatatcatcatccaggtggtcatcgtgttcgcaggcttcctcctgcagggagcagtgctggcccgccaggtgccccaggctgtggtgcgcacggccaacatcctggagtttgctgccaaccacgcctcagtctggatcgccatcctgctcacggttgaccgctacactgccctgtgccaccccctgcaccatcgggccgcctcgtccccaggccggacccgccgggccattgctgctgtcctgagtgctgccctgttgaccggcatccccttctactggtggctggacatgtggagagacaccgactcacccagaacactggacgaggtcctcaagtgggctcactgtctcactgtctatttcatcccttgtggcgtgttcctggtcaccaactcggccatcatccaccggctacggaggaggggccggagtgggctgcagccccgggtgggcaagagcacagccatcctcctgggcatcaccacactgttcaccctcctgtgggcgccccgggtcttcgtcatgctctaccacatgtacgtggcccctgtccaccgggactggagggtccacctggccttggatgtggccaacatggtggccatgctccacacggcagccaacttcggcctctactgctttgtcagcaagactttccgggccactgtccgacaggtcatccacgatgcctacctgccctgcactttggcatcacagccagagggcatggcggcgaagcctgtgatggagcctccgggactccccacaggggcagaagtgtagaggagggggcccagctagggagctcagggtggctcatggccacatgtactggggcctttgaggttgtacccaaaacacgtttatcaacagcttgctttccttgggtgggggtggaggctcctcctttgggtgtggctcccaggtagagaggaggacaacttagccagctcttatgtttgcttcaccagcaatccctatttcctgggaagatgaaagggcactgccaggcacaggctaatagcatcagtgctgtgggcattcctttgcggggggcattttgcctggctcatcgtgaatgccagattaatgttggttgaatggatagaaaaacggacagatggaggccgggtgcggtggctcacgcctgtaatcccagcacgttgggaggctgaggcaggcggatcacgaggtcaggagatcgagaccacagtgaaaccctgtctctactaaaaatacaaaaaattagctggacgcagtggtgggcgcctgtagtcccagctactcgggaggctgaggcaggagaatggcgtgaacccggaaggcggagcttgcggtgagccgagatcccgccactgcactccagcctgggcgacagagtgagactccgtctcaaaaaaaaaaaaa

SEQ ID NO: 4 - DNA, 1466 bp, Mus musculus
  murine islet cell G-protein coupled receptor (IC-GPCR)
ggcacgaggccgtggggcaaataactctccctcacagagccagtgtgagggacacagctggtgatgttgcaaggggctgtgaagatcccaaggaaaagctgctggtgacctgggtgggacccaggacccagaactatgggacttgagggagcagagacagctgtgcagccacaagtgaccctgctgcctacagtcaatggtagtaacccaagatacgatggtgtggatggccactggccagagagcccagagaggtcaccctgtgtggctggtatcatacctgtcatctattacagcgtcctgctgagcctggggctgcctgtcaacgtcctgacttcagtggcactggcccgtcttgctgccaggaccaggaaaccctcctatcactatctcctggcactcacggcttcagatatcgtcacccaagtgatcatcgtcttcgtaggcttcctcctgcagggagctgtgctggcccgccaggtgccccaggccgtggtgcgcacagctaacatcctggagtttgctgccaatcacgcctcagtctggattgcagtcttgttcacggtggatcgctacaatgccctgtgccgccctctacgccatcgggccacctcgtccccaggccggacccaccgcgctattgctgctgtcattggcgttaccctcctgaccggcatcccattctactggtggctggatgtgtggagggatgcagaccctcctagcactatggacaaactcctcaagtgggctcactgcctcatcgtctacttcattccctgcaatgttttcctggtcaccaactctgccatcatccttcggctacggaagagaggccagagagggctgcggccattggtgagcaagagcacagccatcctcctgggtgtcacctccctcttcgccctcctctgggcaccacgtattattgtcatgctgtaccatctgtatgtggcccctgttcaccgggattggagggtccacctggccttggacatagccaacatgttggccatgctcaacacagaggtcaactttggcctctattgcttcatcagcaagactttcagagccactgtccgacaggtcatctgtgatgtccatatggcttgcgccttaaagtcacagccaaagcaaacggtggtggaactcatgctgaagtccgtagggacagagctgtagagctaaaggggccaggctagacatcctcagggccacctgcacagagacctttgcagttgtttggttttggctttggtttccctgtgtagacttggctgtcctccaactcagaggcctctctgcctctgaggcacctatctctgcctccagagtgctagggttaaagatgtgagccaccactgcctgaggagacattcatcaaaatgttaccaactttgcaatctgggggaaaggaaaaggctctggctttgggcaactccgggtaaaaaaaaaaaaaaaaaaaa

SEQ ID NO: 5 - DNA, 22 bp, Artificial Sequence
  Description of Artificial Sequencesense RT-PCR primer for human IC-GPCR gene
gctgagccaggagtttgaaagc

SEQ ID NO: 6 - DNA, 22 bp, Artificial Sequence
  Description of Artificial Sequenceanti-sense RT-PCR primer for human IC-GPCR gene
cacgccacaagggatgaaatag

SEQ ID NO: 7 - DNA, 20 bp, Artificial Sequence
  Description of Artificial Sequencesense RT-PCR primer for mouse IC-GPCR gene
cgggtcttcgtcatgctcta

SEQ ID NO: 8 - DNA, 25 bp, Artificial Sequence
  Description of Artificial Sequenceanti-sense RT-PCR primer for mouse IC-GPCR gene
ggaaagtcttgctgacaaagcagta

SEQ ID NO: 9 - protein, 371 aa, Mus musculus
  mouse pancreatic polypeptide receptor type 2 (PP2)
MEVLTNQPTPNKTSGKSNNSAFFYFESCQPPFLAILLLLIAYTVILIMGIFGNLSLIIIIFKKQREAQNVTNILIANLSLSDILVCVMCIPFTVIYTLMDHWVFGNTMCKLTSYVQSVSVSVSIFSLVLIAIERYQLIVNPRGWKPRVAHAYWGIILIWLISLTLSIPLFLSYHLTNEPFHNLSLPTDIYTHQVACVEIWPSKLNQLLFSTSLFMLQYFVPLGFILICYLKIVLCLRKRTRQVDRRKENKSRLNENKRVNVMLISIVVTFGACWLPLNIFNVIFDWYHEMLMSCHHDLVFVVCHLIAMVSTCINPLFYGFLNKNFQKDLMMLIHHCWCGEPQESYENIAMSTMHTDESKGSLKLAHIPTGI

SEQ ID NO: 10 - protein, 372 aa, Homo sapiens
  human delta opioid receptor
MEPAPSAGAELQPPLFANASDAYPSACPSAGANASGPPGARSASSLALAIAITALYSAVCAVGLLGNVLVMFGIVRYTKMKTATNIYIFNLALADALATSTLPFQSAKYLMETWPFGELLCKAVLSIDYYNMFTSIFTLTMMSVDRYIAVCHPVKALDFRTPAKAKLINICIWVLASGVGVPIMVMAVTRPRDGAVVCMLQFPSPSWYWDTVTKICVFLFAFVVPILIITVCYGLMLLRLRSVRLLSGSKEKDRSLRRITRMVLVVVGAFVVCWAPIHIFVIVWTLVDIDRRDPLVVAALHLCIALGYANSSLNPVLYAFLDENFKRCFRQLCRKPCGRPDPSSFSRAREATARERVTACTPSDGPGGGAAA

SEQ ID NO: 11 - protein, 372 aa, Mus musculus
  mouse delta opioid receptor
MELVPSARAELQSSPLVNLSDAFPSAFPSAGANASGSPGARSASSLALAIAITALYSAVCAVGLLGNVLVMFGIVRYTKLKTATNIYIFNLALADALATSTLPFQSAKYLMETWPFGELLCKAVLSIDYYNMFTSIFTLTMMSVDRYIAVCHPVKALDFRTPAKAKLINICIWVLASGVGVPIMVMAVTQPRDGAVVCMLQFPSPSWYWDTVTKICVFLFAFVVPILIITVCYGLMLLRLRSVRLLSGSKEKDRSLRRITRMVLVVVGAFVVCWAPIHIFVIVWTLVDINRRDPLVVAALHLCIALGYANSSLNPVLYAFLDENFKRCFRQLCRTPCGRQEPGSLRRPRQATTRERVTACTPSDGPGGGAAA

SEQ ID NO: 12 - protein, 399 aa, Mus musculus
  mouse bombesin receptor subtype-3 (BRS-3)
MSQRQSQSPNQTLISITNDTETSSSVAPNDTTHKGWTGDNSPGIEALCAIYITYAGIISVGILGNAILIKVFFKTKSMQTVPNIFITSLAFGDLLLLLTCVPVDATHYLAEGWLFGKVGCKVLSFIRLTSVGVSVFTLTILSADRYKAVVKPLERQPPNAILKTCAKAGGIWIVSMIFALPEAIFSNVYTFQDPNRNVTFESCNSYPISERLLQEIHSLLCFLVFYIIPLSIISVYYSLIARTLYKSTLNIPTEEQSHARKQIESRKRIAKTVLVLVALFALCWLPNHLLYLYHSFTYESYANHSDVPFVIIIFSRVLAFSNSCVNPFALYWLSKTFQQHFKAQLCCLKAEQPEPPLGDIPLNNLTVMGRVPATGSAHVSEISVTLFSGSSAKKGEDKV

SEQ ID NO: 13 - protein, 371 aa, Homo sapiens
  human vasopressin receptor type II (V2R)
MLMASTTSAVPGHPSLPSLPSNSSQERPLDTRDPLLARAELALLSIVFVAVALSNGLVLAALARRGRRGHWAPIHVFIGHLCLADLAVALFQVLPQLAWKATDRFRGPDALCRAVKYLQMVGMYASSYMILAMTLDRHRAICRPMLAYRHGSGAHWNRPVLVAWAFSLLLSLPQLFIFAQRNVEGGSGVTDCWACFAEPWGRRTYVTWIALMVFVAPTLGIAACQVLIFREIHASLVPGPSERPGGRRRGRRTGSPGEGAHVSAAVAKTVRMTLVIVVVYVLCWAPFFLVQLWAAWDPEAPLEGAPFVLLMLLASLNSCTNPWIYASFSSSVSSELRSLLCCARGRTPPSLGPQDESCTTASSSLAKDTSS

SEQ ID NO: 14 - protein, 371 aa, Mus musculus
  mouse vasopressin receptor type II (V2R)
MILVSTTSAVPGALSSPSSPSNSSQEELLDDRDPLLVRAELALLSTIFVAVALSNGLVLGALIRRGRRGRWAPMHVFISHLCLADLAVALFQVLPQLAWDATDRFHGPDALCRAVKYLQMVGMYASSYMILAMTLDRHRAICRPMLAYRHGGGARWNRPVLVAWAFSLLLSLPQLFIFAQRDVGNGSGVFDCWARFAEPWGLRAYVTWIALMVFVAPALGIAACQVLIFREIHASLVPGPSERAGRRRRGHRTGSPSEGAHVSAAMAKTVRMTLVIVIVYVLCWAPFFLVQLWAAWDPEAPLERPPFVLLMLLASLNSCTNPWIYASFSSSVSSELRSLLCCAQRHTTHSLGPQDESCATASSSLMKDTPS

SEQ ID NO: 15 - protein, 363 aa, Homo sapiens [sequence omitted]

SEQ ID NO: 16 - protein, 385 aa, Mus musculus
  mouse somatostatin subtype 5 receptor (SST5)
MCPLVTPSSLAEIHVLWHPEPDSMEPLSLASTPSWNASAASSGSHNWSLVDPVSPMGARAVLVPVLYLLVCTVGLGGNTLVIYVVLRYAKMKTVTNVYILNLAVADVLFMLGLPFLATQNAVSYWPFGSFLCRLVMTLDGINQFTSIFCLMVMSVDRYLAVVHPLRSARWRRPRVAKLASAAVWVFSLLMSLPLLVFADVQEGWGTCNLSWPEPVGLWGAAFITYTSVLGFFGPLLVICLCYLLIVVKVKAAGMRVGSSRRRRSERKVTRMVVVVVLVFVGCWLPFFIVNIVNLAFTLPEEPTSAGLYFFVVVLSYANSCANPLLYGFLSDNFRQSFRKALCLRRGYGVEDADAIEPRPDKSGRPQTTLPTRSCEANGLMQTSRL

SEQ ID NO: 17 - protein, 9 aa, Artificial Sequence
  Description of Artificial Sequenceconsensus peptide for GPCR proteins
TNIYILNLA

SEQ ID NO: 18 - protein, 6 aa, Artificial Sequence
  Description of Artificial Sequenceconsensus peptide for GPCR proteins
MSVDRY

SEQ ID NO: 19 - protein, 8 aa, Artificial Sequence
  Description of Artificial Sequenceconsensus peptide for GPCR proteins
TRMVLVVV

SEQ ID NO: 20 - protein, 4 aa, Artificial Sequence
  Description of Artificial Sequenceconsensus peptide for GPCR proteins
CWAP

SEQ ID NO: 21 - protein, 22 aa, Artificial Sequence
  Description of Artificial Sequencehuman IC-GPCR Transmembrane I domain
IYYSVLLGLGLPVSLLTAVALA

SEQ ID NO: 22 - protein, 23 aa, Artificial Sequence
  Description of Artificial Sequencehuman IC-GPCR Transmembrane II domain
YLLCALTASDIIIQVVIVFAGFL

SEQ ID NO: 23 - protein, 22 aa, Artificial Sequence
  Description of Artificial Sequencehuman IC-GPCR Transmembrane III domain
LEFAANHASVWIAILLTVDRYT

SEQ ID NO: 24 - protein, 22 aa, Artificial Sequence
  Description of Artificial Sequencehuman IC-GPCR Transmembrane IV domain
IAAVLSAALLTGIPFYWWLDMW

SEQ ID NO: 25 - protein, 22 aa, Artificial Sequence
  Description of Artificial Sequencehuman IC-GPCR Transmembrane V domain
AHCLTVYFIPCGVFLVTNSAII

SEQ ID NO: 26 - protein, 22 aa, Artificial Sequence
  Description of Artificial Sequencehuman IC-GPCR Transmembrane VI domain
ILLGITTLFTLLWAPRVFVMLY

SEQ ID NO: 27 - protein, 22 aa, Artificial Sequence
  Description of Artificial Sequencehuman IC-GPCR Transmembrane VII domain
ALDVANMVAMLHTAANFGLYCF

SEQ ID NO: 28 - protein, 22 aa, Artificial Sequence
  Description of Artificial Sequencemouse IC-GPCR Transmembrane I domain
IYYSVLLSLGLPVNVLTSVALA

SEQ ID NO: 29 - protein, 22 aa, Artificial Sequence
  Description of Artificial Sequencemouse IC-GPCR Transmembrane II domain
LLALTASDIVTQVIIVFVGFLL

SEQ ID NO: 30 - protein, 22 aa, Artificial Sequence
  Description of Artificial Sequencemouse IC-GPCR Transmembrane III domain
TANILEFAANHASVWIAVLFTV

SEQ ID NO: 31 - protein, 22 aa, Artificial Sequence
  Description of Artificial Sequencemouse IC-GPCR Transmembrane IV domain
HRAIAAVIGVTLLTGIPFYWWL

SEQ ID NO: 32 - protein, 22 aa, Artificial Sequence
  Description of Artificial Sequencemouse IC-GPCR Transmembrane V domain
AHCLIVYFIPCNVFLVTNSAII

SEQ ID NO: 33 - protein, 22 aa, Artificial Sequence
  Description of Artificial Sequencemouse IC-GPCR Transmembrane VI domain
LLGVTSLFALLWAPRIIVMLYH

SEQ ID NO: 34 - protein, 22 aa, Artificial Sequence
  Description of Artificial Sequencehuman IC-GPCR Transmembrane VII domain
LDIANMLAMLNTEVNFGLYCFI

SEQ ID NO: 35 - protein, 5 aa, Artificial Sequence
  Description of Artificial Sequenceconsensus peptide for IC-GPCR
MGLEG

SEQ ID NO: 36 - protein, 6 aa, Artificial Sequence
  Description of Artificial Sequenceconsensus peptide for IC-GPCR
VTLLPT

SEQ ID NO: 37 - protein, 4 aa, Artificial Sequence
  Description of Artificial Sequenceconsensus peptide for IC-GPCR
HWPE

SEQ ID NO: 38 - protein, 8 aa, Artificial Sequence
  Description of Artificial Sequenceconsensus peptide for IC-GPCR
ERSPCVAG

SEQ ID NO: 39 - protein, 10 aa, Artificial Sequence
  Description of Artificial Sequenceconsensus peptide for IC-GPCR
IPVIYYSVLL

SEQ ID NO: 40 - protein, 5 aa, Artificial Sequence
  Description of Artificial Sequenceconsensus peptide for IC-GPCR
LGLPV

SEQ ID NO: 41 - protein, 7 aa, Artificial Sequence
  Description of Artificial Sequenceconsensus peptide for IC-GPCR
VALARLA

SEQ ID NO: 42 - protein, 10 aa, Artificial Sequence
  Description of Artificial Sequenceconsensus peptide for IC-GPCR
YLLALTASDI

SEQ ID NO: 43 - protein, 36 aa, Artificial Sequence
  Description of Artificial Sequenceconsensus peptide for IC-GPCR
GFLLQGAVLARQVPQAVVRTANILEFAANHASVWIA

SEQ ID NO: 44 - protein, 5 aa, Artificial Sequence
  Description of Artificial Sequenceconsensus peptide for IC-GPCR
TVDRY

SEQ ID NO: 45 - protein, 6 aa, Artificial Sequence
  Description of Artificial Sequenceconsensus peptide for IC-GPCR
SSPGRT

SEQ ID NO: 46 - protein, 6 aa, Artificial Sequence
  Description of Artificial Sequenceconsensus peptide for IC-GPCR
RAIAAV

SEQ ID NO: 47 - protein, 12 aa, Artificial Sequence
  Description of Artificial Sequenceconsensus peptide for IC-GPCR
LLTGIPFYWWLD

SEQ ID NO: 48 - protein, 7 aa, Artificial Sequence
  Description of Artificial Sequenceconsensus peptide for IC-GPCR
LKWAHCL

SEQ ID NO: 49 - protein, 6 aa, Artificial Sequence
  Description of Artificial Sequenceconsensus peptide for IC-GPCR
VYFIPC

SEQ ID NO: 50 - protein, 10 aa, Artificial Sequence
  Description of Artificial Sequenceconsensus peptide for IC-GPCR
VFLVTNSAII

SEQ ID NO: 51 - protein, 8 aa, Artificial Sequence
  Description of Artificial Sequenceconsensus peptide for IC-GPCR
KSTAILLG

SEQ ID NO: 52 - protein, 6 aa, Artificial Sequence
  Description of Artificial Sequenceconsensus peptide for IC-GPCR
LLWAPR

SEQ ID NO: 53 - protein, 5 aa, Artificial Sequence
  Description of Artificial Sequenceconsensus peptide for IC-GPCR
VMLYH

SEQ ID NO: 54 - protein, 16 aa, Artificial Sequence
  Description of Artificial Sequenceconsensus peptide for IC-GPCR
YVAPVHRDWRVHLALD

SEQ ID NO: 55 - protein, 7 aa, Artificial Sequence
  Description of Artificial Sequenceconsensus peptide for IC-GPCR
NFGLYCF

SEQ ID NO: 56 - protein, 12 aa, Artificial Sequence
  Description of Artificial Sequenceconsensus peptide for IC-GPCR
SKTFRATVRQVI

SEQ ID NO: 57 - protein, 200 aa, Artificial Sequence
  Description of Artificial Sequencepoly Gly flexible linker
GGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGG

SEQ ID NO: 58 - protein, 15 aa, Artificial Sequence
  Description of Artificial SequenceC-terminal peptide of human IC-GPCR for raising antibody
CPVMEPPGLPTGAEV

SEQ ID NO: 59 - protein, 15 aa, Artificial Sequence
  Description of Artificial SequenceC-terminal peptide of mouse IC-GPCR for raising antibody
CTVVELMLKSVGTEL